(19) United States
(12) Patent Application Publication
(10) Pub. No.: US 2004/0237144 A1
(43) Pub. Date: Nov. 25, 2004

(54) Anther-specific taa1 genes encoding fatty acyl co-a reductases, and uses thereof

(76) Inventors: Gopalan Selvaraj, Saskatoon (CA); Aiming Wang, London (CA); Qun Xia, Saskatoon (CA); Wenshuang Xie, Saskatoon (CA)

Correspondence Address: KIRBY EADES GALE BAKER, BOX 3432, STATION D, OTTAWA, ON, K1P 6N9, CA

(21) Appl. No.: 10/478,310

(22) PCT Filed: Jun. 7, 2002
(86) PCT No.: PCT/CA02/00834

Related U.S. Application Data
(60) Provisional application No. 60/296,159, filed on Jun. 7, 2001.

(51) Int. Cl. A01H 1/00; C12N 9/04; C07H 21/04; C12N 15/82; C12N 5/04

(52) U.S. Cl. 800/287; 435/69.1; 435/190; 435/320.1; 435/419; 536/23.2

(57) ABSTRACT

The present invention provides isolated and purified polynucleotide sequences encoding fatty acyl Co-A reductase (FAR) enzymes derived from wheat, designated TAA1 genes. The invention encompasses genes that encode FAR enzymes that are useful in the production of transgenic plants and other organisms that comprise increased or otherwise altered levels of fatty alcohols. Such plants may have significant commercial value for the production of fatty alcohols for use in nutritional and pharmaceutical compositions. The invention also provides corresponding TAA1 anther-specific promoters, suitable for the expression of proteins other than FAR enzymes in the anthers and pollen cells of suitably transformed plants.

### 1. FILED OF THE INVENTION

The present invention relates to genes that are specifically expressed in the anthers of plants. More particularly, the present invention relates to genes encoding fatty acyl Co-A reductase enzymes that are required for pollen grain maturation.

### 2. BACKGROUND TO THE INVENTION

There is a significant degree of commercial interest in the development of transgenic plants with altered lipid metabolism, which generate altered or increased yields of lipid products. The development of such modified plants and crops may facilitate the manufacture of nutritional and medicinal products in crops. Therefore, the possibility of successfully generating lipid-modified plants has implications for both the agricultural and pharmaceutical industries.

The metabolic pathways that regulate lipid metabolism in plants are not fully understood. Different regions and organs of a plant generate alternative profiles of lipid products, with certain regions of a plant comprising a greater concentration of lipid products than others. For this reason, the genes involved in lipid metabolism must undergo differential regulation for specific lipid products to be concentrated in particular regions of the plant. Delineation of plant lipid metabolic pathways, and the generation of modified transgenic plants with beneficial characteristics, represents a considerable challenge to those of skill in the art.

The outer surface of pollen grains represents one region of a plant known to harbor higher concentrations of lipid products. The anthers of plants have evolved to coat pollen grains with an oily substance to preserve and increase the viability of the pollen. For this purpose, male gametophyte development, and in particular the interplay between the sporophytic tapetum and gametophytic microspore, is a well-orchestrated process in plants (Goldberg et al., 1993). To date, a number of genes underlying this process have been isolated and characterized. These genes may be grouped into pollen-specific and anther/tapetum-specific genes. The former are usually predominantly expressed during advanced stages of pollen development. The examples include genes encoding cytoskeletal proteins (Kandasamy et al., 1999; Lopez et al., 1996), cell wall-degrading enzymes (Brown and Crouch, 1990; Futamura et al., 2000), pollen allergens (Rafnar et al., 1991) and other genes with unknown functions (Zou et al., 1994). The other group includes genes preferentially expressed in the tapetum at relatively early stages of microsporogenesis. These include genes associated with programmed cell death (Walden et al., 1999), pollen excine formation (Aarts et al., 1997; Fuerstenberg et al., 2000; Koltunow et al., 1990), lipid transfer (Aguirre and Smith, 1993), cell wall-degradation (Bih et al., 1999; Hird et al., 1993; Rubinelli et al., 1998) and unknown functions (Jeon et al., 1999).

The anther tapetum plays a pivotal role in plant gametophyte development (Piffanelli et al., 1998; Shivanna et al., 1997). In addition to breakdown of callus wall around microspore tetrads and supply of nutrients to developing pollens, the essential function of the tapetum is thought to form two extracellular lipid-derived structures (pollen exine and pollen coating) of pollen grains. This assumption is established on the base of the earlier cytological observations, and recent ultrastructural and molecular studies (for recent reviews see Furness and Rudall, 2001; Huysmans et al., 1998; Piffanelli et al., 1998). For example, it has been shown that during the development of the extracellular lipidic structures, the tapetum and not the microspore is the major site of fatty acid biosynthesis (Piffanelli et al., 1997). Mutation of a tapetum-specific gene encoding a putative fatty lipid reductase leads to formation of exine-free pollen and male sterility (Aarts et al., 1997). Recent progress also includes the finding that two tapetum-unique lipidic organelles whose major constituents are neutral esters and polar lipids, upon lysis of the tapetal cells, are discharged into the anther locule and their components contribute to the formation of the lipidic coating of mature pollen grains (Hernández-Pinzón et al., 1999; Piffanelli and Murphy, 1998; Ting et al., 1998; Wu et al., 1997).

These findings substantially facilitate our understanding of the intrinsic link between the tapetal lipid biosynthesis and microspore development. However, the enzymes that catalyze and regulate the biochemical production of these tapetal lipidic compounds have remained unclear. Isolation and characterization of these lipid-specific enzymes would permit an improved understanding of the mechanisms of plant lipid metabolism.

### 3. SUMMARY OF THE INVENTION

It is an object of the present invention to isolate and characterize anther-specific genes involved in lipid metabolism, for the commercial development of useful transgenic plants and plant products.

It is a further object of the present invention to provide a means for modifying lipid metabolism in plants, preferably by increasing or altering the yield of useful lipid-based products in the plants. The present invention further aims to provide a transgenic plant with increased levels of fatty alcohols, which can be harvested for use in the production of, for example, nutritional and pharmaceutical products.

It is another object of the present invention to provide a means of increasing the levels of fatty alcohols in designated regions or organs of a plant, for specific commercial purposes. These commercial purposes may include, but are not limited to, the production of crops with increased pesticide resistance, crops with altered cross-breeding activity, plants with increased levels of lipid products concentrated in regions that permit facile harvesting and extraction.

It is further an object of the present invention to isolate and characterize genes expressed in the tapetum, and their corresponding proteins, that are required for the formation of the outer cell wall of pollen grains during microspore development. In this way, the present invention aims to alter anther-specific properties of a plant to induce, for example, male sterility, and developmental or reproductive modifications that are commercially useful properties. It is still further an object of the present invention to provide a transgenic plant, comprising a construct wherein anther-specific promoters are utilized to generate useful products in the anthers and pollen cells of the transgenic plants.

It is a further object of the present invention to provide isolated recombinant proteins involved in plant lipid metabolism, which can be used in the commercial ex vivo production of fatty alcohols.

The peptides of the present invention, and their corresponding nucleotide sequences, have significant potential for use in the generation of genetically modified plants with altered profiles, or increased or otherwise altered levels of lipid compounds, as well as plant having desirable anther-specific and whole-plant phenotypic modifications.

The inventors of the present application have succeeded in isolating and purifying both the genomic and cDNA sequences of a family of three closely related genes that are predominantly expressed in the tapetum of anthers. The genes were isolated from the bread wheat species ‘Triticum aestivum’, and are designated TAA1a, TAA1b, and TAA1c. The cDNA sequences of these genes have permitted the characterization of the corresponding protein products, which can function as fatty acyl Co-A reductases. Transgenic plants overexpressing a TAA1 gene can comprise higher than normal levels of fatty alcohols. It is considered that similar transgenic plants will have strong potential for the generation of crops capable of producing fatty acid products for agricultural, nutritional and pharmaceutical purposes.

Moreover, isolation of the corresponding genomic DNA sequences for the TAA1 genes has permitted the characterization of the anther-specific TAA1 gene promoters. These promoters are tapetum specific and have significant potential for the generation of constructs for use in transgenic plants, wherein the constructs comprise a gene of choice under the control of the anther-specific promoter. In this way, numerous properties of the plant can be modified to alter, for example, the developmental, reproductive, and aesthetic properties of the plant.

In accordance with a first embodiment of the present invention, there is provided an isolated and purified nucleotide sequence, characterized in that the nucleotide sequence is endogenously expressed in wheat anthers, and encodes a peptide having fatty acyl Co-A reductase (FAR) activity. The present invention therefore provides characterization of a novel family of genes that are involved in lipid metabolism in anthers of plants and encompasses all such corresponding homologous genes.

In an alternative embodiment the present invention provides an isolated and purified nucleotide sequence, characterized in that the nucleotide sequence is selected from:

(a) a TAA1 gene, or a part thereof, or a complement thereof; and

(b) a nucleotide sequence having at least 50% identity to a peptide encoded by a TAA1 gene, or a part thereof, or a complement thereof; the nucleotide sequence encoding a protein or a part thereof, that alters lipid metabolism in a transgenic plant exogenously expressing said nucleotide sequence.

Preferably, the isolated and purified nucleotide sequence is selected from:

(a) SEQ ID NO: 1, 3, or 5, or a part thereof, or a complement thereof; and

(b) a nucleotide sequence having at least 50% identity to a peptide encoded by a SEQ ID NO: 1, 3, or 5, or a part thereof, or a complement thereof; the nucleotide sequence encoding a protein or a part thereof, that alters lipid metabolism in a transgenic plant exogenously expressing said nucleotide sequence. More preferably, the nucleotide sequence has at least 70%, more preferably 90%, more preferably 95% and most preferably 99% identity to a peptide encoded by a SEQ ID NO: 1, 3, or 5, or a part thereof, or a complement thereof. In this way, the nucleotide sequences of the present invention include TAA1 homologous genes from species of plants other than wheat, as well as closely related wheat homologues, polymorphisms and mutated variants of the genes. The invention further encompasses nucleotide sequences that will bind to SEQ ID NOS: 1, 3, or, 5 under stringent hybridization conditions, including nucleotide sequences suitable for use as hybridization probes, PCR primers and DNA sequencing primers.

In further embodiments, the present invention also encompasses isolated and purified peptides, or parts thereof, encoded by TAA1 genes, or possible variants of the TAA1 genes disclosed herein. Such peptides may be used in the production of pharmaceutical or nutritional agents as appropriate.

The present invention further encompasses expression cassettes and constructs comprising TAA1 gene sequences and variants, complements, or parts thereof. Preferably, the expression cassettes and constructs include a TAA1 gene sequence open reading frame operably linked to a promoter for expression of the TAA1 gene product, or part or variant thereof. Preferably, the expression cassettes and constructs of the present invention are suitable for transformation into plants. In this way, transgenic plants having altered lipid metabolism or altered lipid content can be generated. More preferably, the altered lipid metabolism or altered lipid content at least partly occurs within the anthers and/or pollen of the transgenic plant.

The transgenic plants of the present invention therefore include plants expressing the nucleotide sequences disclosed herein, and homologues and variants thereof, thereby increasing, decreasing or changing the lipid content of the plant compared to an unmodified plant. More preferably, the change in lipid content may specifically relate to the fatty alcohol content of the plant, and more preferably the fatty alcohol content of the anthers and/or pollen of the plant. The transgenic plants of the present invention include species of a woody plants, non-woody plants, and grasses, as well as plants selected from the group consisting of crucifer crops, tobacco, wheat, corn, sugar cane, and apple.

In an alternative embodiment, the transgenic plants of the present invention may include constructs wherein the TAA1 gene or part or variant thereof is under the control of an organ-specific promoter. In this way, the promoter can direct the expression of the nucleotide sequence to affect a particular organ or organs of the plant. The transgenic plants of the present invention may exhibit one or more modified characteristics compared to an unmodified plant including, but not limited to: increased pest resistance; male sterility; reduced height; reduced internode spacing; increased resistance to wind damage; reduced growth rate; altered cross-pollination specification; increased fruit or nut aesthetic appeal; delayed vegetative development; and delayed propagative development.

The transgenic plants of the present invention may contain constructs characterized in that the nucleotide sequence expressed is oriented for antisense expression from the construct, thereby causing a reduction in the levels of fatty acyl Co-A reductase compared to an unmodified plant, and a corresponding decrease in the levels of fatty alcohols present in the plant.

The present invention further encompasses an isolated and purified nucleotide sequence, characterized in that the nucleotide sequence is selected from:

(a) SEQ ID NO: 7, 8, or 9 or a complement thereof; and

(b) a nucleotide sequence that can hybridize to SEQ ID NO: 7, 8, or 9 or a complement thereof under stringent hybridization conditions. Therefore, the invention encompasses the corresponding genomic DNA sequences for the TAA1 family of genes, including promoter sequence disclosed in SEQ ID NOS: 7, 8, and 9, or TAA1 promoter sequence obtained by chromosome walking a genomic DNA library for 5′ (and 3′) untranslated regions of the TAA1 genomic DNAs. Furthermore, in alternative embodiments the invention includes nucleotide sequences for use as hybridization probes, PCR primers or DNA sequencing primers, that bind to the TAA1 sequences under stringent hybridization conditions. Preferably, the promoters of the present invention can be used to direct the expression of a gene unrelated to fatty acyl Co-A reductases in the anthers and pollen grains of transgenic plants. Most preferably, the promoter of the present invention may comprise of genomic DNA sequence of about 1.6 kb upstream from the start codon of SEQ ID NO: 8.

In additional embodiments, the present invention includes constructs comprising TAA1 promoter sequences in operative association with an open reading frame, or a part thereof or a complement thereof, for use in modifying anther, tapetum or pollen metabolism. The constructs may be transformed into plants to generate transgenic plants with altered characteristics. For example, the invention encompasses transgenic plants transformed with a construct having a TAA1 promoter or part thereof in operative association with an anther or pollen inactivating gene, wherein expression of the open-reading frame induces male sterility of the transgenic plant. Alternatively, the open-reading frame may encode a transposase, and expression of the open-reading frame may induce an increased rate of genomic DNA rearrangement in anther or pollen cells of the transgenic plant. Alternatively, the open-reading frame may encode a peptide suitable for use as a nutritional or pharmaceutical agent, the peptide being expressed in anthers or pollen of the transgenic plant. Alternatively, the open-reading frame may encode a peptide required for the production of a nutritional or pharmaceutical agent, or a protein that inhibits the production and/or accumulation of an unwanted substance selected from the group consisting of a toxin, and an allergen, or a peptide for altering the cross-pollination specification of the transgenic plant. Alternatively, the open reading frame may be oriented for antisense expression within the construct, thereby inducing antisense repression of endogenous gene expression within the anthers, tapetum or pollen of the transgenic plant.

The present invention further provides, in alternative embodiments, for a means for generating fatty alcohols that may be used as nutritional or pharmaceutical agents. The fatty alcohols may be purified from extracts of the transgenic plants using techniques that are well known in the art. Preferably, the fatty alcohols generated by the transgenic plants of the present invention include Octacosanol; a fatty alcohol known to produce health benefits including enhances physical endurance and reproductive health. Moreover, in another preferred embodiment, the transgenic plants of the present invention may be used to generate fatty alcohols for the washing and cleaning industry. In alternative embodiments, the transgenic plants of the present invention may bear fruit with increased levels of fatty alcohols, wherein the fruit include wax derived from the fatty alcohols to help preserve the fruit and improve the aesthetic appeal of the fruit, thereby improving shelf life. The increased levels of wax production in the plants of the present invention are further predicted to confer enhanced properties such as reduced rates of moisture loss, and increased resistance to pests.

The invention further encompasses the fatty alcohols derived or extracted from the transgenic plants or other transformed organisms (e.g. bacteria) of the present invention, and their use, for example as a wax, as a cleaning agent, as a cosmetic agent, as a dermatological agent, as a pharmaceutical agent, or as a nutritional agent.

The invention further encompasses pharmaceutical and nutritional compositions and agents comprising the plant extracts and fatty alcohols obtained from the transgenic plants of the present invention, as well as methods for treating or preventing a medical condition, or for providing a dietary supplement, by the administration of the plant extracts or fatty alcohols of the present invention.

The invention further encompasses method for the production and isolation of fatty alcohols, characterized in that the method comprises the steps of: transforming an organism with a construct comprising a TAA1 gene sequence, or part thereof, or complement thereof in accordance with the present invention; growing or propagating said organism containing said construct; and extracting said fatty alcohols from said organism. Preferably, the organism is an E. coli bacterium, such that recombinant E. coli comprising increased or altered levels of fatty alcohols may be cultured and harvested. In an alternative embodiment, the organism may comprise a plant or a plant embryo, preferably a tobacco plant or tobacco plant embryo, that is induced to express the construct and generate increased or altered levels of fatty alcohols. Similarly, such transgenic plants may be grown and/or propagated thereby allowing plant extracts to be harvested and fatty alcohols to be purified by standard techniques.

The invention further encompasses a method of inducing dwarfism in a plant,

characterized in that the method comprises the steps of:

transforrning a plant cell, plant embryo or plant with a construct according to the present invention; and

growing or propagating said plant cell, plant embryo, or plant, thereby generating a plant expressing a DNA sequence encoded by said construct, said plant having a reduced size compared to an unmodified plant.

### 4. BRIEF DESCRIPTION OF THE DRAWINGS

FIG. 1(a) schematically illustrates the genomic organization of TAA1a, TAA1b, and TAA1c. Triangles and rectangles represent introns and exons, respectively. The length (bp: base pair) of each intron and exon is shown above and in the corresponding triangle and rectangle. The stippled and hatched triangles indicate a very long intron and an intron with alternative insertion position in TAA1b, respectively. The putative translation start (AUG) and stop (TGA) codons, and 5′ and 3′ UTR (untranslational region) are given. (A)n represents a poly(A) tail. For clarity, cDNA sequences and introns are not drawn to scale.

FIG. 1(b) provides a genomic DNA blot analysis of different wheat species. Molecular size makers are indicated at left in kilobases. Sources of DNA are shown. Total DNA (10 μg) was digested with EcoRI (E), BanzHI (B) and HindIII (H), separated on a 1% agarose gel, blotted onto a nylon membrane, probed with the coding region of TAA1a, and visualized by exposure to an x-film.

FIG. 2(a) demonstrates anther-specific expression of TAA1. R, roots; S, stems; L, leaves; A, anthers; O, ovary; G, glume and pilea. Northern blot analysis of TAA1 expression in wheat. Total RNA (about 5 μg) purified from root, stem, leaf, anther, ovary, and pilea and glume was loaded. A 0.7 kb fragment of the TAA1a cDNA resulting from 5′ RACE was used as a probe. The estimated size of hybridizing RNA species is shown to the left side. Underneath is the same blot hybridized with a 28S rRNA probe.

FIG. 2(b) demonstrates anther-specific expression of TAA1. R, roots; S, stems; L, leaves; A, anthers; O, ovary; G, glume and pilea. RT-PCR amplification of cDNA derived from different wheat tissues. Underneath is the same cDNA amplified with a pair of primers to a glyceraldehyde-3-phosphate dehydrogenase gene (GPD).

FIG. 3 In situ RNA hybridization. The cross-sections of wheat flower buds were hybridized with TAA1 anti-sense and sense transcript. Hybridization was shown by the formation of a dark bluish precipitate. Solid arrow head: tapetum; unfilled arrow head: micropore; m, microspores; ov, ovary; and ps, pollen sac. Scale bars=100 μm.

(a) probed with a TAA1a sense transcript.

(b) probed with a TAA1 antisense transcript.

(c) sectioned at stage pre-meiosis, probed with a TAA1 antisense transcript.

(d) sectioned at stage young microspore, probed with a TAA1 antisense transcript.

(e) sectioned at stage vacuolated microspore, probed with a TAA1 antisense transcripts.

FIG. 4 Immunocytochemical detection of the TAA1 protein on the wheat anthers. The sections were immunoblotted with either pre-immune serum or TAA1a antiserum. Positive antibody recognition was shown by the formation of bluish deposits. M, microspores; ps, pollen sac, arrow heads, tapetum. Scale bars=100 μm.

(a) immunoreacted with pre-immune serum (control).

(b) with TAA1a antiserum.

(c) an enlarged pollen sac of (a) (control).

(d) sectioned at the young microspore stage and immunoreacted with TAA1a antiserum.

(e) sectioned at the vacuolated microspore stage.

FIG. 5(a). Amino acid sequence comparison and phylogenetic analysis of TAA1. Pair-wise alignment of the amino acid sequence of TAA1a with that of FAR according to Pearson and Lipman (1998). A represents gaps which are introduced to allow the best matches. The dashes in FAR indicate the identical residues to TAA1a. Two potential transmembrane helixes predicted by Metz et al. (2000) are underlined.

FIG. 5(b). Phylogenetic analysis of TAA1 and its related genes. The sequences of all related genes were obtained from public databases and refer to the following: FAR, the jojoba acyl coenzyme A reductase (accession no. AF149917); MS2-like; a predicted gene from Arabidopsis (accession no. AB012244); MS2, the Arabidopsis male sterility 2 gene (accession no. S33804); B-MS2, the Brassica MS2 gene (accession no. T08096).

FIG. 6(a) Fatty alcohols in transgenic seeds and E. coli. Fatty alcohol content in the tobacco seeds transformed with the Napin-TAA1a chimeric gene. The amounts of fatty alcohols obtained from GC analysis were normalized against the internal standard beta-sitosterol. The y-axis of the graph illustrates percentage ‘FA’ of the relative amounts of fatty alcohols to beta-sitosterol (%). Line 723-0-D was transformed with the control vector. All the remaining (477-0-4, 477-0-18, 477-0-2, and 477-0-10) were the Napin-TAA1a transgenic lines.

FIG. 6(b) Gas chromatography (GC) analysis of fatty alcohol amounts and compositions in bacterial cells without ((i) upper graph), or with ((ii) lower graph) expression of TAA1a. t=retention time in minutes, and CL=chain length of fatty alcohol standards.

FIG. 7. Over-expression of TAA1 results in significant dwarfism in transgenic tobacco. Vector: transgenic plants containing NPTII resistant gene only; 35S::TAA1: transgenic containing both NPTII resistant gene and 35S::TAA1a chimeric gene. In this example, TAA1 was over-expressed constitutively. (a) Three-week old seedlings in MS medium. (b) Plants three weeks after transplanting in a greenhouse. (c) Plants two months after transplanting in greenhouse.

FIG. 8 Transient expression assay of TAA1 promoter specificity. Hand cross-section of Daylily flower buds were bombarded with microprojectiles coated with either the CaMV35S-uidA (a) or TAA1-uidA chimeric genes (b). 35S-GUS transient expression was observed in anther walls, filaments, and petals (a). In contrast, TAA1-GUS transient expression limited in microspores and tapetum (arrow) (b). an: Anther, f: Filament, m: microspores, pe: petal, ps: pollen sac. Scale bars=1 mm.

FIG. 9 GUS expression pattern in transgenic tobacco anthers. (a) and (b) show GUS assays on hand cross-sections of anthers at different developmental stages of a transgenic plant containing a TAA1-uidA chimeric gene. (a) at the tetrad stage and (b) at the microspore separation stage. (c) and (d) show paraffin cross-sections of anthers of transgenic plants containing a 35S-uidA chimeric gene and a TAA1-uidA chimeric gene, respectively. aw: anther wall; cn: connective tissue; ep: epidermis; m: microspores; t: tapetum. Scale bars=200 μm.

### 6. GLOSSARY OF TERMS

Amplification of DNA/amplified DNA: “amplified DNA” refers to the product of nucleic-acid amplification of a target nucleic-acid sequence. Nucleic-acid amplification can be accomplished by any of the various nucleic-acid amplification methods known in the art, including the polymerase chain reaction (PCR). A variety of amplification methods are known in the art and are described, inter alia, in U.S. Pat. Nos. 4,683,195 and 4,683,202, and in Innis et al. (eds.), PCR Protocols: A Guide to Methods and Applications, Academic Press, SanDiego, 1990.

Construct: A construct comprises a vector and a DNA molecule operatively linked to the vector, such that the vector and operatively linked DNA molecule can be replicated and transformed as required.

Expression: The generation of a protein product derived from a DNA sequence encoding the protein, comprising a combination of transcription and translation.

Homologous: DNA or peptide sequences exhibiting similarity to another DNA or peptide sequences in terms of the chemical nature, order and position of the individual residues relative to one another in the sequence. For the purposes of this application, unless stated otherwise homology is characterized according to BLAST search results, wherein a best-fit sequence alignment is obtained. In this way, sequences comprising residues that are similar or identical may be aligned, and gaps provided as necessary. Homology is therefore expressed as a percentage of similarity or identity, wherein similarity encompasses both similar and identical residues. Unless stated otherwise, all BLAST searches were carried out using default parameters: e.g. gaps permitted, E-value=1, organism selected as required, filter for low complexity, standard genetic code, BLOSUM62 general purpose matrix; for more information see http://www.ncbi.nlm.nih.gov/Education/BLASTinfo/tut1.html.

Identity: Comparison of homologous DNA or peptide sequences provides identification of residues that are identical in the same relative position of the sequence, following best fit alignment. For the purposes of this application, unless stated otherwise, homology, best fit alignment and identity are calculated according to BLAST search results (BLAST searching is available, for example, from the following website: http://www.ncbi.nlm.nih.gov/BLAST/). Identity is provided as a percentage, indicating the percentage of residues that are identical along the sequences under comparison, excluding regions of gaps between the aligned sequences. BLAST searching permits a standard alignment configuration to automatically take into account regions of gaps or truncations between sequences, thereby providing a ‘best fit’ alignment.

Isolated: A nucleotide or peptide is “isolated” if it has been separated from other cellular components (nucleic acids, liquids, carbohydrates, and other nucleotides or peptides) that naturally accompany it. Such a nucleotide or peptide can also be referred to as “pure” or “homogeneous” or “substantially” pure or homogeneous. Thus, a nucleotide or peptide which is chemically synthesized or recombinant is considered to be isolate. A nucleotide or peptide is isolated when at least 60-90% by weight of a sample is composed of the nucleotide or peptide, preferably 95% or more, and more preferably more than 99%. Protein purity or homogeneity is indicated, for example, by polyacrylamide gel electrophoresis of a protein sample, followed by visualization of a single peptide band upon staining the polyacrylamide gel; high-performance liquid chromatography; or other conventional methods. The peptides of the present invention can be purified by any of the means known in the art. Various methods of protein purification are described, e.g., in Guide to Protein Purification, in Deutscher (ed.), Meth. Enzymol. 185, Academic Press, San Diego, 1990; and Scopes, Protein Purification: Principles and Practice, Springer Verlag, New York, 1982.

Operably linked: two nucleotide sequences are operable linked if the linkage allows the two sequences to carry out their normal functions relative to each other. For instance, a promoter region would be operably linked to a coding sequence if the promoter were capable of effecting transcription of that coding sequence, and the coding sequence encoded a product intended to be expressed in response to the activity of the promoter.

Organ: A specific region of a plant defined in terms of structure and function, for example, a stem, a leaf, an anther, a pollen grain, or a root.

Promoter: A recognition site on a DNA sequence or group of DNA sequences that provides at least one expression control element for a gene encoding a polypeptide, and to which RNA polymerase specifically binds and initiates RNA synthesis (transcription) of the gene.

Stringent conditions, or stringent hybridization conditions: includes reference to conditions under which a probe will hybridize to its target sequence, to a detectably greater degree than other sequences (e.g. at least 2-fold over background). Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH. The Tm is the temperature (under defined ionic strength and pH at which 50% of a complementary target sequence hybridizes to a perfectly matched probe. Typically, stringent conditions will be those in which the salt concentration is less than about 1.0 M Na ion, typically about 0.01 to 1.0 M Na ion concentration (or other salts) at pH 7.0 to 8.3 and the temperature is at least about 30° C. for short probes (e.g. 10 to 50 nucleotides) and at least about 60° C. for long probes (e.g. greater than 50 nucleotides). Stringent conditions may also be achieved with the addition of destabilizing agents such as formamide. Exemplary low stringency conditions include hybridization with a buffer solution of 30% formamide, 1 M NaCl, 1% SDS at 37° C., and a wash in 2×SSC at 50° C. Exemplary high stringency conditions include hybridization in 50% formamide, 1 M NaCl, 1% SDS at 37° C., and a wash in 0.1×SSC at 60° C. Hybridization procedures are well-known in the art and are described in Ausubel et al.,(Ausubel F. M., et al., 1994, Current Protocols in Molecular Biology, John Wiley & Sons Inc.).

Transformation: Modification of a cell by the introduction of exogeneous DNA sequence (e.g. a vector or recombinant DNA molecule).

Transgenic: A cell or organism derived from a process of cellular transformation, wherein the cell or organism comprises the introduced exogenous DNA molecule not originally present in a non-transgenic cell or organism.

Transgenic plant: A plant or progeny thereof derived from a transformed plant cell or protoplast, wherein the plant DNA contains an introduced exogenous DNA molecule not originally present in a native, non-transgenic plant of the same strain. The terms “transgenic plant” and “transformed plant” have sometimes been used in the art as synonymous terms to define a plant whose DNA contains an exogenous DNA molecule. However, it is thought more scientifically correct to refer to a regenerated plant or callus obtained from a transformed plant cell or protoplast as being a transgenic plant.

Vector: A DNA molecule capable of replication in a host cell and/or to which another DNA segment (or insert) can be operatively linked so as to bring about replication of the attached insert. A plasmid is an exemplary vector. Moreover, a vector may include promoter sequence to facilitate expression of an open reading frame present in the DNA insert. All vectors used for the present application were generated by the inventors, with the exception of: T/A vectors (Invitrogen), pRSET A (Invitrogen), phagemids (Stratagene), pRD400 and pRD410 (Datla et al. 1992), pHS724 (Huang et al., 2000), pJOY43 (Nair et al., 2000).

### 7. DETAILED DESCRIPTION OF THE PREFERRED EMBODIMENTS

To support this application, two deposits of biological material have been made under the Budapest Treaty regarding Deposits of Biological Material. The deposits were made at the International Depository Authority of Canada, Bureau of Microbiology, Health Canada, Winnipeg, Manitoba, Canada, on Jun. 7, 2001, under accession numbers IDAC 070601-2 and IDAC 070601-1. The deposits both comprise E.coli bacterial cells, strain DH5α, transformed with constructs comprising DNA sequence of the present invention. Deposit number IDAC 070601-2 consists of DH5α cells transformed with pAMW133 comprising the full length coding region of TAA1a cDNA. Deposit number IDAC 070601-1 consists of DH5α cells transformed with pAMW170 comprising the promoter region of the TAA1b gene.

### Nucleotide Sequences Encompassed by the Present Invention

The present invention provides a polynucleotide molecule comprising nucleotide sequences derived from the TAA1 family. The genetic sequences encompassed by the present invention include, but are not limited to, TAA1a cDNA (SEQ ID NO: 1), TAA1b cDNA (SEQ ID NO: 3), TAA1c cDNA (SEQ ID NO: 5), TAA1a genomic DNA (SEQ ID NO: 7), TAA1b genomic DNA (SEQ ID NO: 8), and TAA1c genomic DNA (SEQ ID NO: 9).

Whilst the present invention discloses polynucleotide sequences for three closely related genes, homologous nucleotide sequences encoding peptides with significant amino acid sequence identity to those encoded by SEQ ID NOS: 1, 3, 5, 7, 8, and 9, can be readily obtained in accordance with the teachings of the present application (and references disclosed herein), and are encompassed within the scope of the present invention. In this regard, nucleotide sequences of the present invention can be used to produce (degenerate) nucleotide probes, for the purposes of screening cDNA and genomic DNA libraries of various plant species. Related techniques are well understood in the art, for example as provided in Sambrook et al., Molecular Cloning: A Laboratory Manual, Cold Spring Harbor Press, Cold Spring Harbor, N.Y.(1989). In this way, sequences homologous to those of the present application are readily obtainable. For this reason, it is the intention of the present invention to encompass polynucleotide molecules comprising DNA sequences that encode peptides with significant sequence identity to those disclosed in the present application, wherein SEQ ID NOS: 1, 3, 5, 7, 8, or 9, or parts thereof, may be utilized as polynucleotide probes to search for and isolate homologous polynucleotide molecules. Moreover, polynucleotides encoding proteins with significant sequence identity to those of the present application are expected give rise to similar protein products with similar biochemical characteristics, to those described in the present application. Indeed, such techniques were used by the inventors to isolate the various TAA1 cDNA and genomic DNA homologous sequences disclosed herein. More details in this regard are provided in the examples.

The present invention therefore encompasses DNA sequences obtained by techniques known in the art for isolating homologous DNA sequences, wherein the techniques utilize degenerate oligonucleotide probes derived from a sequence selected from SEQ ID NO:1, 3, 5, 7, 8, and 9, or parts thereof. The degree of amino acid sequence identity will vary for each identified sequence. It is the intention of the present invention to encompass polynucleotide sequences comprising at least 50% sequence identity with regard to the peptide sequences encoded by the corresponding polynucleotides. Without wishing to be bound by theory, it is generally expected in the art that enzymes with at least 50% identity can have enzymatic activities that are similar in scope. In this regard, the essential structural features of the enzyme are preserved to scaffold the conformation of the catalytic site of the enzyme. Therefore, the present invention encompasses polynucleotide molecules derived by screening genomic and cDNA libraries of plant types including wheat and other species, using degenerate DNA probes derived from the sequences disclosed in the present application. Such species include, but are not restricted to: rye, barley, rice and other grasses, and monocots such as maize, and lily.

The present invention also encompasses polynucleotide sequences obtained by screening DNA libraries using degenerate oligonucleotide probes derived from the polynucleotides of the present invention, wherein the sequences encode peptides comprising at least 70% amino acid sequence identity to peptides encoded by SEQ ID NOS: 1, 3, 5, 7, 8, and 9. In this regard, homologous proteins with at least 70% predicted amino acid sequence identity are expected to encompass proteins with similar fatty acyl Co-A reductase activity as those defmed by the present invention, but possibly with altered substrate specificity. Such proteins may be derived from related species of plant.

The present invention also encompasses polynucleotide sequences encoding peptides comprising at least 90%, 95% or 99% sequence identity to the peptides encoded by SEQ ID NOS: 1, 3, 5, 7, 8, and 9. This class of related proteins is intended to include close gene family members with very similar or identical catalytic activity. In addition, peptides with 90%, 95% or 99% amino acid sequence identity may be derived from functional homologues of similar species of plant, or from directed mutations to the sequences disclosed in the present application.

### Isolation of TAA1 cDNA and Genomic DNA Homologues

With the provision of several TAA1 cDNA and genomic DNAs, the polymerase chain reaction (PCR) may now be utilized in a preferred method for isolating further TAA1 homologous nucleotide sequences from wheat and other species of plant. PCR amplification of the TAA1 cDNA sequence may be accomplished either by direct PCR from a plant cDNA library or by Reverse-Transcription PCR (RT-PCR) using RNA extracted from plant cells as a template. Methods and conditions for both direct PCR and RT-PCR are known in the art and are described in numerous standard textbooks. Similarly, the TAA1 genomic sequences may be amplified directly from genomic DNA extracted from plants, or from plant genomic DNA libraries. Amplification may be used to obtain the full length cDNA or genomic sequence, or may be used to amplify selected portions of these molecules (for example for use in antisense constructs).

Moreover, the well known technique of chromosome walking can be readily used to isolate regions of genomic DNA that are 5′ or 3′ to the coding region of the gene. The technique of chromosome walking is described, for example, in Sambrook et al., Molecular Cloning: A Laboratory Manual, Cold Spring Harbor Press, Cold Spring Harbor, N.Y.(1989). The present disclosure includes analysis of a region upstream of to the TAA1b genomic DNA start codon, that is suitable for use as an anther specific promoter.

The selection of PCR primers will be made according to the portions of the TAA1 nucleic acids which are to be amplified, including full-length TAAI clones. Variations in amplification conditions may be required to accommodate primers of differing lengths; such considerations are well known in the art.

Oligonucleotides which are derived from the TAAI nucleic acid sequences described herein, and which are suitable for use as PCR primers to amplify additional TAA1 nucleic acid sequences are encompassed within the scope of the present invention. Preferably, such oligonucleotide primers will comprise a sequence of 15-20 consecutive nucleotides of the TAA1 nucleic acid sequences. To enhance amplification specificity, primers comprising at least 20-30 consecutive nucleotides of these sequences may also be used.

With the provision herein of the TAA1 nucleic acid sequences, the cloning by standard methodologies of corresponding cDNAs and genes from other ecotypes and plant species, as well as polymorphic forms of the disclosed sequences is now enabled. Thus, the present invention includes methods of isolating a nucleotide sequence encoding a TAA1 enzyme from a plant. Both conventional hybridization and PCR amplification procedures may be utilized to clone such sequences. Common to both of these techniques is the hybridization of probes or primers derived from the disclosed TAAI nucleic acid sequences to a target nucleotide preparation, which may be, in the case of conventional hybridization approaches, a cDNA or genomic library or, in the in the case of PCR amplification, extracted genomic DNA, mRNA, a cDNA library or a genomic library.

Direct PCR amplification may be performed on cDNA libraries prepared from the plant species in question, or RT-PCR may be performed using mRNA extracted from the plant cells using standard methods. PCR primers will comprise at least 15 consecutive nucleotides of the TAA1 nucleic acid sequences. One of skill in the art will appreciate that sequence differences between the disclosed TAA1 nucleic acid sequences and the target gene to be amplified may result in lower amplification efficiencies. To compensate for this, longer PCR primers or lower annealing temperatures may be used during the amplification cycle. Where lower annealing temperatures are used, sequential rounds of amplification using nested primer pairs may be necessary to enhance specificity.

### Generation of TAA1 variants and mutants

For conventional hybridization techniques, the hybridization probe is preferably labeled with a detectable label such as a radioactive label, and the probe is of at least 20 nucleotides in length. As is well known in the art, increasing length of hybridization probes tends to give enhanced specificity. The labeled probe derived from, for example, the TAA1 cDNA sequence may be hybridized to a plant cDNA or genomic library and the hybridization signal detected using means known in the art. The hybridizing colony or plaque (depending on the type of library used) is then purified and the cloned sequence contained in that colony or plaque isolated and characterized.

It will also be understood to a person of skill in the art that site-directed mutagenesis techniques are readily applicable to the polynucleotide sequences of the present invention. Related techniques are well understood in the art, for example as provided in Sambrook et al., Molecular Cloning: A Laboratory Manual, Cold Spring Harbor Press, Cold Spring Harbor, N.Y.(1989). In this regard, the present invention teaches the isolation and characterization of the DNA sequences as provided as SEQ ID NOS: 1, 3, 5, 7, 8, and 9. However, the present invention is not intended to be limited to these specific sequences. Numerous directed mutagenesis techniques would permit the non-informed technician to alter one or more residues in the nucleotide, thus changing the subsequently expressed polypeptide sequences. Moreover, commercial ‘kits’ are available from numerous companies that permit directed mutagenesis to be carried out (available for example from Promega and Biorad). These include the use of plasmids with altered antibiotic resistance, uracil incorporation and PCR techniques to generate the desired mutations. The mutations generated may include point mutations, deletions and truncations as required. The present invention is therefore intended to encompass corresponding mutants of the TAA1 cDNA and genomic DNA sequences disclosed in the present application.

The mutated variants of the sequences of the present application are predicted to include enzymes with reduced or increased fatty acyl Co-A reductase activity, as well as altered substrate specificity. Such mutants may confer advantageous properties to subsequently transformed transgenic cell lines and plants. For example, a transgenic plant comprising a construct overexpressing an inactive mutant of the enzymes of the present invention can be expected to have a significantly altered profile of lipid constituents, including a possible reduction in fatty alcohol content. In contrast, the expression of mutant fatty acyl Co-A reductase enzymes with increased catalytic turnover are expected to give rise to transgenic plants with an high level of fatty alcohols. Mutant fatty acyl Co-A reductase enzymes with altered substrate specificity will likely be useful in altering the relative quantities of lipid metabolism products generated in a correspondingly transformed plant, or altering the distribution of the lipid metabolism products within the organs of the plant.

### Generation of Constructs Comprising TAA1 Sequence

The polynucleotide sequences of the present invention can be ligated into suitable vectors before transfer of the genetic material into plants. For this purpose, standard ligation techniques that are well known in the art may be used. Such techniques are readily obtainable from any standard textbook relating to protocols in molecular biology, and suitable ligase enzymes are readily available from commercial sources. A number of recombinant vectors suitable for stable transfection of plant cells or for the establishment of transgenic plants have been described, which are also readily available from commercial sources. Typically, plant transformation vectors include one or more cloned plant genes (or cDNAs) under the transcriptional control of 5′ and 3′ regulatory sequences and a dominant selectable marker. Such plant transformation vectors typically also contain a promoter regulatory region (e.g., a regulatory region controlling inducible or constitutive, environmentally- or developmentally-regulated, or cell- or tissue-specific expression), a transcription initiation start site, a ribosome binding site, an RNA processing signal, a transcription termination site, and/or a polyadenylation signal.

As noted above, the particular arrangement of the TAA1 nucleic acid in the transformation vector will be selected according to the expression of the nucleic acid desired.

Where enhanced fatty alcohol synthesis is desired, the TAA1 nucleic acid may be operably linked to a constitutive high-level promoter such as the CaMV 35S promoter. Modification of fatty alcohols synthesis may also be achieved by introducing into a plant a transformation vector containing a variant form of the TAA1 nucleic acid, for example a form which varies from the exact nucleotide sequence of the TAA1 nucleic acid, but which encodes a protein that retains the functional characteristic of the TAA1 protein, i.e., fatty acyl Co-A reductase activity.

In contrast, a reduction of fatty alcohol synthesis may be obtained by introducing antisense constructs based on the TAA1 nucleic acid sequence into plants. For antisense suppression, the TAA1 nucleic acid is arranged in reverse orientation relative to the promoter sequence in the transformation vector. The introduced sequence need not be the full length TAA1 nucleic acid, and need not be exactly homologous to the TAA1 nucleic acid. Generally, however, where the introduced sequence is of shorter length, a higher degree of homology to the native TAA1 sequence will be needed for effective antisense suppression. Preferably, the introduced antisense sequence in the vector will be at least 30 nucleotides in length, and improved antisense suppression will typically be observed as the length of the antisense sequence increases. Preferably, the length of the antisense sequence in the vector will be greater than 100 nucleotides. Transcription of an antisense construct as described results in the production of RNA molecules that are the reverse complement of MRNA molecules transcribed from the endogenous TAA1 gene in the plant cell. Although the exact mechanism by which antisense RNA molecules interfere with gene expression has not been elucidated, it is believed that antisense RNA molecules bind to the endogenous mRNA molecules and thereby inhibit translation of the endogenous mRNA or trigger the degradation of MRNA, or inhibit transcription by causing methylation of the gene. A variation of the antisense suppression includes RNAi strategy as published in the literature under various names such as double stranded (dsRNA) RNA suppression.

Suppression of endogenous TAA1 gene expression can also be achieved using ribozymes. Ribozymes are synthetic RNA molecules that possess highly specific endoribonuclease activity. The production and use of ribozymes are disclosed in U.S. Pat. No. 4,987,071 to Cech and U.S. Pat. No. 5,543,508 to Haselhoff, which are hereby incorporated by reference. The inclusion of ribozyme sequences within antisense RNAs may be used to confer RNA cleaving activity on the antisense RNA, such that endogenous MRNA molecules that bind to the antisense RNA are cleaved, which in turn leads to an enhanced antisense inhibition of endogenous gene expression.

Constructs in which the TAA1 nucleic acids (or variants thereon) are over-expressed may also be used to obtain co-suppression of the endogenous TAA1 gene in the manner described in U.S. Pat. No. 5,231,021 to Jorgensen. Such co-suppression (also termed sense suppression) does not require that the entire TAA1 nucleic acid be introduced into the plant cells, nor does it require that the introduced sequence be exactly identical to the TAA1 nucleic acid. However, as with antisense suppression, the suppressive efficiency will be enhanced as (1) the introduced sequence is lengthened and (2) the sequence similarity between the introduced sequence and the endogenous TAA1 gene is increased.

### Transformation of TAA1 Constructs

The present invention also encompasses a plant cell transformed with a nucleotide sequence of the present invention, and as well as plants derived from propagation of the transformed plant cells. Numerous methods for plant transformation have been developed, including biological and physical, plant transformation protocols. See, for example, Miki et al., “Procedures for Introducing Foreign DNA into Plants” in Methods in Plant Molecular Biology and Biotechnology, Glick, B. R. and Thompson, J. E. Eds. (CRC Press, Inc., Boca Raton, 1993) pages 67-88. In addition, expression vectors and in vitro culture methods for plant cell or tissue transformation and regeneration of plants are available. See, for example, Gruber et al., “Vectors for Plant Transformation” in Methods in Plant Molecular Biology and Biotechnology, Glick, B. R. and Thompson, J. E. Eds. (CRC Press, Inc., Boca Raton, 1993) pages 89-119.

The following are examples, and are not limiting:

A. Agrobacterium-mediated Transformation: One method for introducing an expression vector into plants is based on the natural transformation system of Agrobacterium. See, for example, Horsch et al., Science 227: 1229 (1985). A. tumefaciens and A. rhizogenes are plant pathogenic soil bacteria which genetically transform plant cells. The Ti and Ri plasmids of A. tumefaciens and A. rhizogenes, respectively, carry genes responsible for genetic transformation of the plant. See, for example, Kado, C. I., Crit. Rev. Plant. Sci.10: 1 (1991). Descriptions of Agrobacterium vector systems and methods for Agrobacterium-mediated gene transfer are provided by Gruber et al., supra, Miki et al., supra, and Moloney et al., Plant Cell Reports 8: 238 (1989). Bechtold et al., C. R. Acad. Sci. Paris Life Sciences, 316:1194-9 (1993).

B. Direct Gene Transfer: Several methods of plant transformation, collectively referred to as direct gene transfer, have been developed as an alternative to Agrobacterium-mediated transformation. A generally applicable method of plant transformation is microprojectile-mediated transformation wherein DNA is carried on the surface of microprojectiles measuring 1 to 4. mu.m. The expression vector is introduced into plant tissues with a biolistic device that accelerates the microprojectiles to speeds of 300 to 600 m/s which is sufficient to penetrate plant cell walls and membranes. Sanford et al., Part. Sci. Technol. 5: 27 (1987), Sanford, J. C., Trends Biotech. 6: 299 (1988), Klein et al., Bio/Technology 6: 559-563 (1988), Sanford, J. C., Physiol Plant 79: 206 (1990), Klein et al., Biotechnology 10: 268 (1992). See also U.S. Pat. No. 5,015,580 (Christou, et al), issued May 14, 1991; U.S. Pat. No. 5,322,783 (Tomes, et al.), issued Jun. 21, 1994.

C. Other methods: Another method for physical delivery of DNA to plants is sonication of target cells. Zhang et al., Bio/Technology 9: 996 (1991). Alternatively, liposome or spheroplast fusion have been used to introduce expression vectors into plants. Deshayes et al., EMBO J., 4: 2731 (1985), Christou et al., Proc Natl. Acad. Sci. U.S.A. 84: 3962 (1987). Direct uptake of DNA into protoplasts using CaCl₂ precipitation, polyvinyl alcohol or poly-L-ornithine have also been reported. Hain et al., Mol. Gen. Genet.199: 161 (1985) and Draper et al., Plant Cell Physiol.23: 451 (1982). Electroporation of protoplasts and whole cells and tissues have also been described. Donn et al., In Abstracts of VIIth International Congress on Plant Cell and Tissue Culture IAPTC, A2-38, p 53 (1990); D'Halluin et al., Plant Cell 4:1495-1505 (1992) and Spencer et al., Plant Mol. Biol. 24: 51-61 (1994).

Following transformation of target cell(s) or tissues, expression of the above-described selectable marker genes allows for preferential selection of transformed cells, tissues and/or plants, using regeneration and selection methods now well known in the art.

The foregoing methods for transformation would typically be used for producing a transgenic variety. The transgenic variety could then be crossed, with another (non-transformed or transformed) variety, in order to produce a new transgenic variety.

Alternatively, a genetic trait which has been engineered into a particular line using the foregoing transformation techniques could be moved into another line using traditional backcrossing techniques that are well known in the plant breeding arts. For example, a backcrossing approach could be used to move an engineered trait from a public, non-elite variety into an elite variety, or from a variety containing a foreign gene in its genome into a variety or varieties which do not contain that gene. As used herein, “crossing” can refer to a simple X by Y cross, or the process of backcrossing, depending on the context. Once a transgenic plant has been established, it is important to determine the phenotype of the seeds of the plant.

Accordingly, in a preferred embodiment of the invention a method is provided for modifying the seed of a plant comprising the steps of:

(a) introducing into a plant cell capable of being transformed and regenerated into a whole plant a construct comprising, in addition to the DNA sequences required for transformation and selection in plants, a nucleotide sequence in accordance with the nucleotide sequences encompassed by the present invention, operably linked to a promoter; and

(b) recovery of a plant which contains the nucleotide sequence.

The present invention therefore encompasses the transformation of a variety of plant species, including woody, non-woody, fruit bearing and grass species, with the DNA sequences disclosed. Particularly preferred varieties include crucifier crops, tobacco, wheat, corn, sugar cane, and apple. The present invention is particularly considered to be useful in the generation of modified fruits such as apples, since increased expression of fatty acyl Co-A reductase enzymes of the present invention is expected to increase the fatty alcohol concentration in the fruits, thus providing the fruits with a more waxy texture, and a more aesthetically pleasing coating.

### Fatty Acid Analysis and Purification

Once a transgenic plant has been established, it is important to determine the fatty alcohol content of the plant, or various plant organs. For this purpose, several techniques are known in the art to for the analysis of the chemical content of plant material, and in particular, the lipid and fatty alcohol content of the plant. These techniques include Gas Chromatography (GC), high performance liquid chromatography, and MS-GC, as well as other techniques that are familiar to those of skill in the art. Moreover, the fatty alcohol products may be extracted from the plant by any one of a range of techniques that are well known in the art for the purposes of lipid extraction.

One example of fatty alcohol purification from plant materials is outlined in the Experimental Procedures where fatty alcohols. were purified for Gas Chromatography analysis. An alternative method was published by Miwa, T. K. 1971. Journal of The American Oil Chemists' Society. 48:259-264, in relation to jojoba oil analysis.

### Production of Recombinant TAA1 Protein using Heterologous Expression Systems

Many different expression systems are available for expressing cloned nucleic acid molecules. Examples of prokaryotic and eukaryotic expression systems that are routinely used in laboratories are described in Chapters 16-17 of Sambrook et al. (1989), which are herein incorporated by reference. Such systems may be used to express a TAA1 protein or derivatives thereof at high levels to facilitate purification and functional analysis of the enzyme. Apart from permitting the activity of the enzyme to be determined (which is particularly useful to assess the activity of homologous and derivative proteins), heterologous expression facilitates other uses of the purified enzyme. For example the purified enzyme produced by recombinant means may be used to synthesize fatty alcohols and other fatty acid metabolites in vitro, particularly radio- or fluorescent-labeled forms of fatty alcohols and metabolites. These molecules may be used as tracers to determine the location in plant tissues and cells of fatty alcohols and their metabolites. The purified recombinant enzyme may also be used as an immunogen to raise enzyme-specific antibodies. Such antibodies are useful as both research reagents (such as in the study of fatty alcohol regulation in plants) as well as diagnostically to determine expression levels of the enzyme in agricultural products, including pollen.

By way of example only, high level expression of the TAA1 protein may be achieved by cloning and expressing the cDNA in yeast cells using the pYES2 yeast expression vector (Invitrogen, San Diego, Calif.). Secretion of the recombinant TAA1 from the yeast cells may be achieved by placing a yeast signal sequence adjacent to the TAA1 coding region. A number of yeast signal sequences have been characterized, including the signal sequence for yeast invertase. This sequence has been successfully used to direct the secretion of heterologous proteins from yeast cells. Alternatively, the enzyme may be expressed at high level in standard prokaryotic expression systems, such as E. coli.

### Homology of TAA1 Nucleic Acids to Other Plant Genes

It is predicted that the TAA1 genes are the first anther-specific genes in wheat to be reported. TAA1 appears to be related to the jojoba FAR (reported by Metz et al., 2000). Both have an Mᵣ of ˜58,000, and share ˜44% aa identity and ˜63% similarity. This FAR is the only biochemically characterized enzyme for which a deduced structure is available. It belongs to the category of alcohol-forming FARs that produces fatty alcohols directly from fatty acyl CoA, one of the two penultimate substrates in wax biosynthesis (Kolattukudy, 1970). The other category of fatty acyl CoA reductases is smaller and produces aldehydes (Mᵣ˜30 kDa) (Vioque and Kolattukudy, 1997), and thus TAA1 is further distinct from these. In jojoba, FAR was isolated from developing seeds. It is not clear if it is also expressed in the anther and other tissues.

The predicted TAA1 gene products share a lesser, yet significant homology with two known anther-specific genes, the Arabidopsis MS2 gene (Aarts et al., 1997) and the Brassica MS2 gene (Aarts et al., 1997; Hodge et al., 1992). Both of them also share significant homology with FAR (˜40% identity and 59% similarity). Any functional implication of TAA1 and MS2 relationship must be considered in light of the observation that TAA1 shows a greater relationship to the jojoba FAR. The Arabidopsis MS2 gene is required for pollen development (Aarts et al., 1993; Aarts et al., 1997). The post-meiotic, tapetal expression of MS2 in Arabidopsis and that of TAA1 in wheat are almost identical. Thus, despite the deviation in the deduced primary structures, both appear to be functionally similar; While a partial redundancy of MS2 function has not been ruled out (Aarts et al., 1993; Aarts et al., 1995), the Arabidopsis genome database (Arabidopsis thaliana geneome CD) was searched using the MS2 aa sequence. Excluding the MS2 itself, seven hypothetical proteins including two named male sterility 2-like, four called acyl coA reductase-like, and one putative protein were found. These proteins are 37-42% identical to and 59-65% similar with the MS2 aa sequence. These proteins range from 409 to 527 aa in length and also share aa homology to TAA1 at similar levels. Moreover, BLASTN searches using the MS2 cDNA did not retrieve any more sequences than itself, indicating that at the nt level, the MS2 does not share significant homology with these predictive genes. In contrast, these genes seem to be more closely related to each other as they conserve significant similarities both at the aa level and the nt level (data not shown). However, it is not know if these genes function in pollen development.

Aarts et al. (1993) identified a short segment of homology between a wheat mitochondrial sequence (Spencer et al., 1992) and the Arabidopsis MS2 at the deduced aa sequence level. This (93/153 aa) occurred over two stretches of the mitochondrial sequence with an unrelated sequence flanked by hallmarks of nuclear splice junctions that connected the two parts. Since splicing in mitochondrial transcripts follows a different scheme, Aarts et al. (1997) proposed that the mitochondrial sequence is of nuclear origin that had recently migrated to the organellar genome. Were this the case, there should be a greater homology between the nt sequence of TAA1, a wheat genomic sequence, and the wheat mitochondrial sequence. Instead, there was no significant homology even though the deduced TAA1a protein did show a similar rate of homology to a discontiguously translated polypeptide of the mitochondrial sequence at the same region as MS2 (93/153 aa). Therefore, the corresponding mitochondrial sequence is more likely due to the biochemical convergence in evolution than a genealogical relationship.

This application-provides convincing evidence that TAA1 is an FAR. FAR converts fatty acyl coA to fatty alcohol (Kolattukudy, 1970; Kolattukudy and Rogers, 1986; Lardizabal et al., 2000; Metz et al., 2000). This is the first plant tapetum-specific gene which is enzymatically identified to be associated with lipid and wax biosynthesis. Pollen grains are coated with two layers of lipidic structures, i.e., the pollen outer wall (exine) and the pollen outmost coating (tryphine) that is overlaid on exine. Sporopollenin is the major constituent of exine and contains metabolites derived from long chain fatty acids and phenylpropanoids (reviewed by Scott, 1994). Although long chain fatty lipids seem to be definitely required for the synthesis of sporopollenin, how sporopollenin is polymerized and what precursors participate in the polymerization still remain unclear. In crucifer plants such as Arabidopsis and Brassica, the deposition of the exine takes place from the completion of meiosis II, through the tetrad and ring-vacuolate stages, to the time of the first pollen mitosis (Piffanelli et al., 1998). During this process, the tapetum is performing very active lipid biosynthesis (Piffanelli et al., 1998). So, it is logically assumed that the tapetum plays a major role in exine formation. Prior to this report, the only functionally characterized anther tapetum-specific gene was the Arabidopsis MS2 gene, whose expression pattern is concomitant with the formation of the pollen outer cell wall. Disruption of the MS2 gene with a transposon results in male sterility. Pollen development in the ms2 mutant shows most dramatic defect upon release from tetrads (Aarts et al., 1997). These pollens lack exine. Though the enzymatic nature of its encoded polypetide has yet to be identified, it shares 40% aa sequence identity with FAR. The present application includes evidence that TAA1 has the MS2 expression pattern during formation of microspore exine and its encoded polypeptide has fatty alcohol forming capacity. Thus, as proposed by Scott (1994) and Aarts et al. (1997), fatty alcohols, the TAA1 or MS2 enzymatic products are likely to be the precursors for sporopollenin polymerization.

The outmost layer of the pollen grain is the pollen coating or tryphine derived from two tapetum-specific lipid-rich organelles, elaioplasts and tapetosomes (Hernández-Pinzón et al., 1999; Piffanelli et al., 1998; Ting et al., 1998; Wu et al., 1997). The former is a plastid with triacylgycerol (TAG) and neutral esters, and the later is a lipid body containing neutral lipids including TAG and wax esters, and also oleosin-like proteins. The main functions of tryphine include pollen-stigma recognition and subsequent pollen hydration (Piffanelli et al., 1998). Analysis of tryphine lipid fractions shows that the tryphine lipids contain TAG, triterpene esters, sterol esters and very long-chain wax esters (Bianchi et al., 1990; Preuss et al., 1993). Of these compounds, long-chain lipids and linear waxes are thought to be essential for the functions of the pollen coating (Lemieux, 1996; Mariani and Wolters-Arts, 2000; Negruk et al., 1996; Preuss et al., 1993). This is in agreement with the previous observations that wax defective mutants such as the cer1, cer2, cer3, cer6, cer8 and cer10 mutants in Arabidopsis exhibit conditional male sterility (Hannoufa et al., 1993; Koornneef et al., 1989). Of these mutants, some have tryphine with smaller lipid droplets than wild-type pollen and some have tryphine without lipids. Recently, a complementation experiment of the cer6 mutants by transgenically expressing the CER6 gene has shown that the two phenotypic effects, i.e. wax defection and male sterility cannot be rescued equally (Fiebig et al., 2000). CER6 is identical to CUT1 encoding an enzyme responsible for elongation of fatty acyl CoA, and silencing CUT1 induces waxless stem and male sterility (Millar et al., 1999). Interestingly, some fertility-restored lines (CER6 transformants) of the cer6 mutnats still show wax-defective stem. Analysis on very long fatty lipids reveals that low amounts of long fatty lipids are sufficient for pollen hydration and germination, suggesting that this remarkable difference results from the different requirements for CER6 activity on stems and the pollen coating (Fiebig et al., 2000). Apart from the ingenious very long chain lipids, a class of exogenous TAGs which is absent in the pollen coating of Arabdopsis also can rescue the fertility of an Arabidopsis wax-defective mutant (Wolters-Arts et al., 1998). These findings raise a possibility that the tryphine function is dependent on the nature of the mixture of lipids including TAG, very long fatty acids and waxes in the tryphine. The composition of and the relative amounts of each species of the lipid pool rather than a single macromolecule determine a functional pollen coating.

Apart from their.presence in pollen lipidic structures (Bianchi et al., 1990), linear wax esters are also ubiquitously present in the plant cuticle (Piffanelli et al., 1998; Post-Beittenmiller, 1996), indicating plant encoded FAR genes have evolved divergent expression mechanisms. Indeed, an alcohol-forming FAR was previously purified from pca leaves (Vioque and Kolattukudy, 1997). Since the lipid composition of tryine is significantly different from that of the cuticle and even that of the intracellular contents of the pollen grains in the same plants (Bianchi et al., 1989; Bianchi et al., 1990; Piffanelli et al., 1997), there must be a mechanism by which the wax biosynthesis is spatially and temporally regulated. Searches of the Arabidopsis genome database identify 7 FAR-like hypothetical proteins excluding the MS2. It would be possible to investigate if these FAR-like genes contribute to divergent lipid biosynthesis in plants. Alternatively, isolation and characterization of more FAR genes will definitely assist in understanding this complicate regulation mechanism.

The present application provides convincing evidence that TAA1 can reduce long chain acyl CoA to fatty alcohols. Fatty alcohols can be further esterified with fatty acids to generate linear wax esters. Transgenic plants that overexpress TAA1 proteins via their natural (tapetum specific) promoters are predicted to have an increased consumption of fatty acyl CoA by TAA1 in the tapetum, which in turn may impact upon lipid-related biosynthesis in the anthers. In this regard, alteration of the lipid composition in the tapetun by TAA1 may be also required for the tryphine development to assure its recognition and hydration function. Corresponding effects upon lipid metabolism are predicted to occur if the TAA1 protein is overexpressed in a plant organ other than the tapetum. For this reason, the present invention encompasses DNA constructs, and the corresponding transgenic plants transformed with the constructs, wherein the over- or under-expression of TAA1-like proteins gives rise to altered lipid metabolism by virtue of an abnormal level of fatty acyl Co-A. Such modifications to lipid metabolism can have profound effects upon phenotype, developmental, reproductive, growth and structural characteristics of the plant. Moreover, the nature and impact of these effects are expected to depend upon the extent of TAA1 expression, and the localisation of TAA1 expression to specific plant organs. Both of these factors are regulated in part the strength and specificity of the promoter.

Specific embodiments of the present invention are illustrated by way of the following examples:

### EXAMPLE 1

### The TAA1 Group of Genes in Wheat—Identification and Structural Characteristics of the cDNA Clones

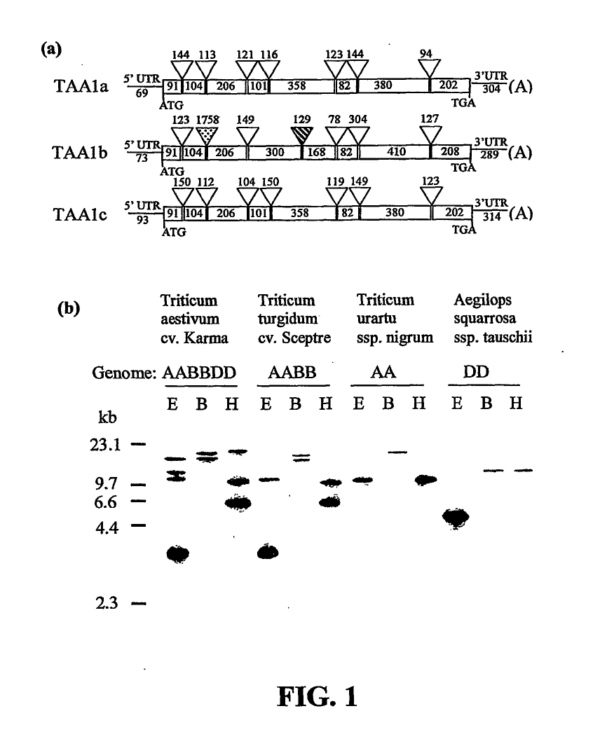

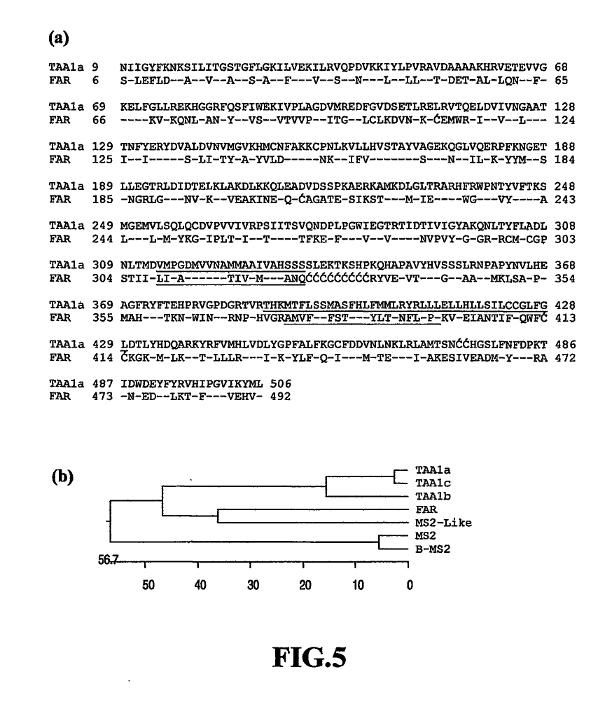

RT-PCR experiments were conducted using an anther-specific cDNA library with primers specific for the rice PS1 gene (Zou et al., 1994). At a moderate annealing temperature of 43° C. a 0.7-kb amplicon was obtained from mRNA isolated from anther but not from root, stem, leaf, glume and pilea tissues. DNA sequencing of the amplicon en masse gave an unambiguous result indicating that the PCR product was composed of a homogeneous sequence within the detection limits of sequencing reaction. Since this amplicon was specific to anther mRNA, a full-length cDNA clone encompassing the amplicon sequence was obtained by 5′-and 3′-RACE. On probing an anther cDNA library with the full-length cDNA, 12 clones were identified and these were grouped into three groups according to their restriction pattern (data not shown). The longest clones from each group were studied further. 5′-RACE experiments did not produce further extensions, suggesting that the longest clones were full-length cDNA clones and hereafter referred as TAA1a, TAA1b and TAA1c (SEQ ID NOS: 1, 3, and 5 respectively). Both TAA1a cDNA (GenBank accession number AJ459249) and TAA1c cDNA (GenBank accession number AJ459253) clones have a predicted open reading frame (ORF) of 1524 nucleotides (nt) encoding 507 amino acids (aa) but with different lengths of 5′ UTRs (TAA1a: 69 nt and TAA1c: 93 nt) (FIG. 1a). The TAA1b cDNA (GenBank accession number AJ459251) ORF has a slightly larger ORF (1569 nt) encoding 522 amino acids and a 5′ UTR of 73 nt. The putative polyadenylation signals, AATAA or TATAA, were found in the 3′ UTR of all three TAA1 cDNAs. Database analysis on the deduced aa sequences encoded by these three genes revealed that they all shared similarity to the fatty acyl-coenzymeA reductase (FAR) gene of jojoba (Metz et al., 2000) and the A. thaliana MS2 gene (Aarts et al., 1993) (see later re.: FIG. 5). On further examination, the primers initially used contained at their 3′ end a high level of identity to two portions of the cDNA clone encompassing the 0.7 kb cDNA fragment.

### EXAMPLE 2

### The TAA1 Group of Genes in Wheat—Identification and Structural Characteristics of the Genomic DNA Clones

The genomic counterparts of the entire coding region of all three TAA1 cDNAs were obtained by PCR amplification of the genomic DNA with primers based on the cDNAs. The genomic DNA sequences for TAA1a, TAA1b, and TAA1c are shown in SEQ ID NOS. 7, 8, and 9 respectively. The TAA1a genomic DNA has been assigned Genbank accession number AJ459250, the TAA1b genomic DNA has been assigned GenBank accession number AJ459252, and the TAA1c genomic DNA has been assigned GenBank Accession number AJ459254 (genomic sequences submitted to GenBank include only the genomic DNA regions encompassing coding sequence).

Nucleotide sequence analysis of the amplicons showed 7 introns interrupting the coding regions in all three genes. The length and composition varied among the three genes. The most significant difference was in the length of the second intron (1758 nt in TAA1b, but only 113 in TAA1a and 112 in TAA1c). The position of the 4^{th} intron in TAA1b also deviated substantially (FIG. 1a).

### EXAMPLE 3

### TAA1 Genes are Likely to Exist as Single Copy per Haploid Genome

Southern blot analysis of the wheats of different genetic constitution—namely, AABBDD, AABB, AA and DD—revealed that the TAA1 genes are likely to exist as single copy per haploid genome. This interpretation was possible because of the choice of restriction enzymes that either did not cut within the coding sequence or cut only rarely and the use of the entire coding sequence of TAA1a cDNA as the probe. Despite this probe coverage, only one hybridization band was observed in the diploids, two in the tetraploid and no more than 4 in the hexaploid (FIG. 1b). Assuming that introns of any paralogs would have caused a restriction polymorphism at this level, these results are consistent with a single copy gene per haploid genome equivalent. The presence of four bands in the hexaploid blot is due to restriction within a TAA1 gene (data not shown).

### EXAMPLE 4

### TAA1 Expression is Confined Primarily to the Anther Tapetum and Associated with Microsporogenesis—Molecular Biology Studies

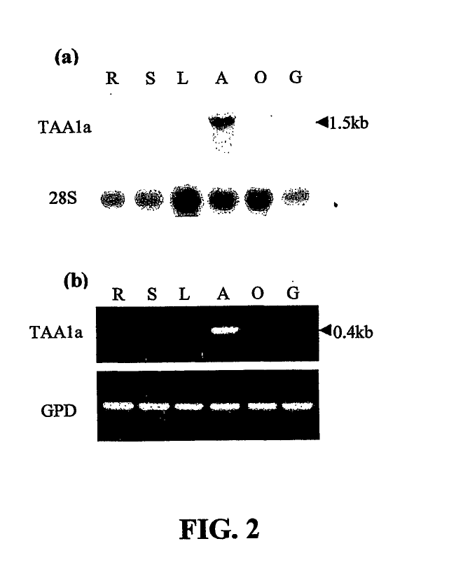

The expression pattern of TAA1 in wheat was determined by probing RNA blots with the 0.7 kb amplicon of TAA1a. The TAA1a probe strongly hybridized only to the anther mRNA, and did not show any hybridization with the root, stem, leaf, ovary or glume transcripts (FIG. 2a). For enhanced detection, RT-PCR of these RNA preparations was done with a primer pair designed to cover parts of two exons with an intron in-between so as to discriminate amplicons of mRNA (˜0.4 kb) and genomic DNA origin (˜0.8 kb). A strong band of ˜0.4 kb was detected in the anther sample, and there was a weak signal in the stem but not in other samples (FIG. 2b). The identity of the amplicon was confirmed by nt sequencing. Thus, TAA1 gene expression is specific to the anther tissue.

### EXAMPLE 5

### TAA1 Expression is Confined Primarily to the Anther Tapetum and Associated with Microsporogenesis—In situ Hybridisation Studies

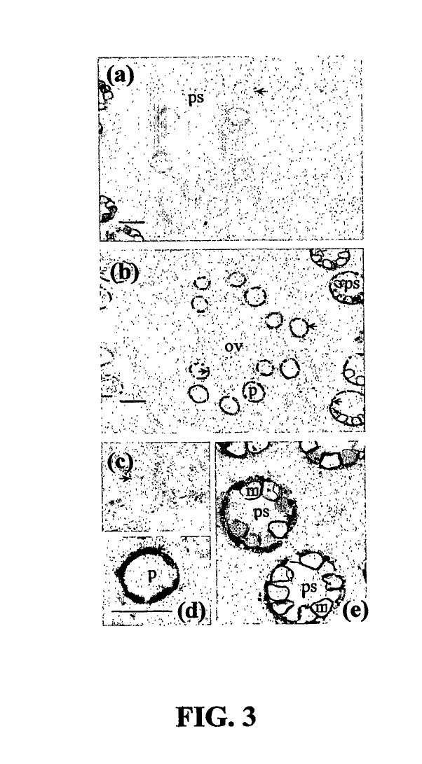

In situ RNA hybridization of transverse sections of flowers at various stages of development with an antisense probe (FIG. 3c and d) showed expression in the tapetum but not in ovary, epidermis, connective tissues and the filament at various ages. With a sense probe, no significant signal was detected (FIG. 3a). Generally, wheat anther development follows seven stages (Saini, 1984; Dorion et al., 1996; Lalonde et al., 1997). These stages include pre-meiosis, meiosis, young microspore, vacuolated microspore (microspores irregularly shaped and in contact with the tapetum, and microspore wall and pore formation in progress), PGM1 (microspore nucleus divides to form vegetative and generative nuclei), PGM2 (tapetal cell walls break down), and mature pollen grain. The onset of TAA1 transcription was not evident until the microspore separation occurred at a stage corresponding to the presence of a young microspore. From then on, TAA1 mRNA was predominantly distributed in tapetum cells and to a lesser extent in some microspores (FIG. 3b and c). TAA1 was strongly expressed at the vacuolated microspore stage when microspore cell walls were evident. The disappearance of TAA1 transcripts coincided with tapetum degeneration (PGM2 stage). Thus, TAA1 transcription is confined (with the exception of weak expression in stem) to anthers, and within anthers it is localized in the tapetum from the point of the formation of young microspores to the degeneration of the tapetum (PGM2 stage).

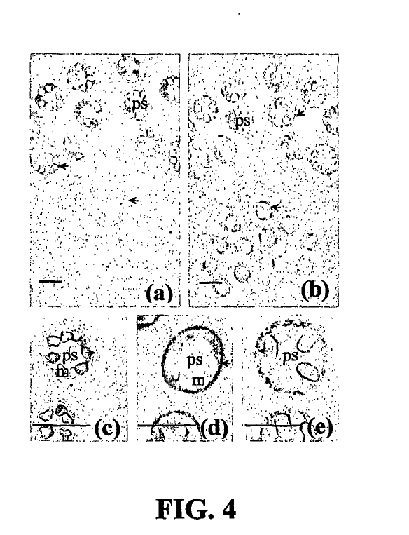

To further test whether the TAA1 gene product is also dominantly localized in the tapetum, the TAA1 specific poly-clonal antibodies were generated and used in in situ immunochemical analysis (FIG. 4) of floral tissues. The results were consistent with RNA in situ-hybridization, suggesting the TAA1 protein was indeed produced in the tapetum as young microspores developed to the PGM2 stage. There was also evidence of its production, albeit at a much lower level, in microspore cells at this stage. Other floral tissues did not show any distinctive signals. Taken together these data indicate that the TAA1 gene products are associated with microspore development.

### EXAMPLE 6

### The TAA1 Gene Product Shares Homology with the Jojoba Seed-borne FAR and the Arabidopsis Anther-specific MS2-encoded Protein

Database analysis of TAA1 was performed to search homologous genes and explore its potential function. While BLAST searches (conducted in accordance with Altschul et al., 1997) of GenBank with the ORF of TAA1a, TAA1b and TAA1c did not identify any significant homologs, the deduced aa sequence (>84% similarity, >74% identity among the three TAA1 peptide sequences) had homologs from A. thaliana (MS2 and MS2-like; Aarts et al., 1993), Brassica napus (B-MS2; Hodge et al., 1992) and Simmondsia chinensis (fatty acyl CoA reductase gene (FAR); Metz et al., 2000). TAA1 gene products were found to be most similar to the jojoba (S. chinensis) FAR and the Arabidopsis putative MS2-like protein (61-65% similarity and 42-46% identity), and to a lesser extent to the MS2 and the Brassica MS2 (54-57% similarity and 35 to 39% identity). Notably, MS2 and its functional ortholog from B. napus (89% identical to MS2; Aarts et al., 1997) have an additional stretch of 117-aa at their amino-terminal region in comparison with TAA1, MS2-like and FAR. Of all these related gene products, only FAR has been biochemically characterized (Metz et al., 2000). Thus, even though the wheat TAA1 gene products share a developmental connotation with the Arabidopsis MS2 gene, TAA1 bears a greater relationship to the characterized jojoba FAR (FIG. 5a and b). The latter is associated with accumulation of storage lipids in seeds and thus presumably inconsequential to anther development. The pair-wise alignment of the aa sequence of the TAA1a-encoded polypeptides to that of the jojoba FAR was carried out to explore conserved domains. There are two consensus regions containing more than 12 consecutive amino acids (FIG. 5a). Interestingly, these two regions are located at the two predicted transmembrane helices (FIG. 5a) (Metz et al., 2000). Further examination of the corresponding regions on the other related gene products revealed that the first putative transmembrane helix of FAR is globally conserved while the second one is not (data not shown).

### EXAMPLE 7

### Accumulation of Fatty Alcohols in the TAA1 Transgenic Tobacco Seeds, and Influence of TAA1 Expression on Plant Phenotype

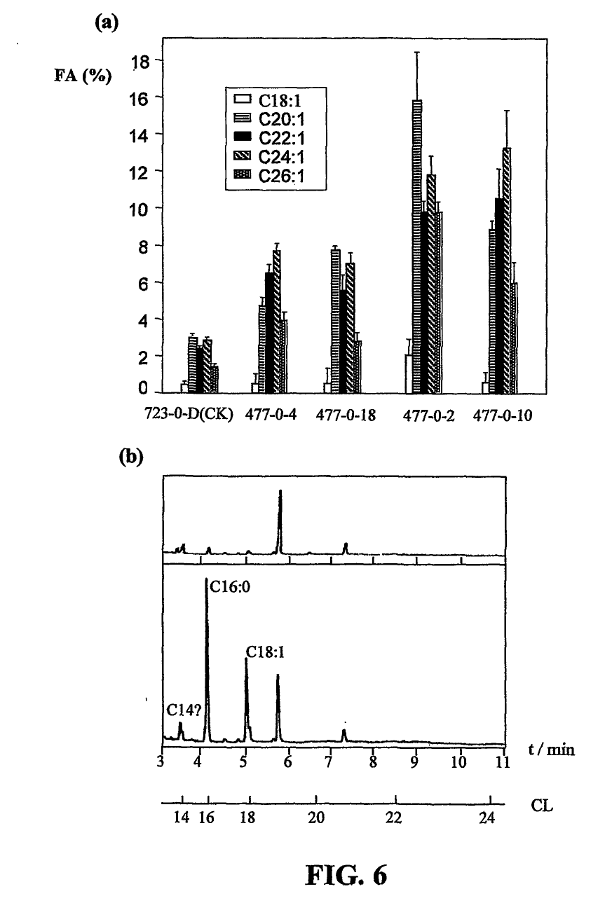

To initiate functional characterization of the TAA1 gene in planta, the TAA1a cDNA was cloned into a binary vector under the control of a napin promoter. The napin-TAA1a chimeric gene was transformed into tobacco. Tobacco seeds contain 30-43% oil and are rich in fatty acids. The potential TAA1 substrates, fatty acyl coA, are actively synthesized in the developing seeds (Frega et al., 1991). Total fatty alcohols were extracted from transgenic seeds. GC analysis on fatty alcohol contents and compositions showed that the TAA1-encoded enzyme significantly modified the pathway of fatty alcohol synthesis in the napin-TAA1 transgenic seeds. The amounts of the five major fatty alcohols, i.e., C18:1, C20:1, C22:1, C24:1 and C24:1 increased by 8.75%-357.47%, 57.78%-426.8.95%, 130.96%-307.72%; 145.00%-361.49% and 99.89%-5929.47%, respectively (FIG. 6a).

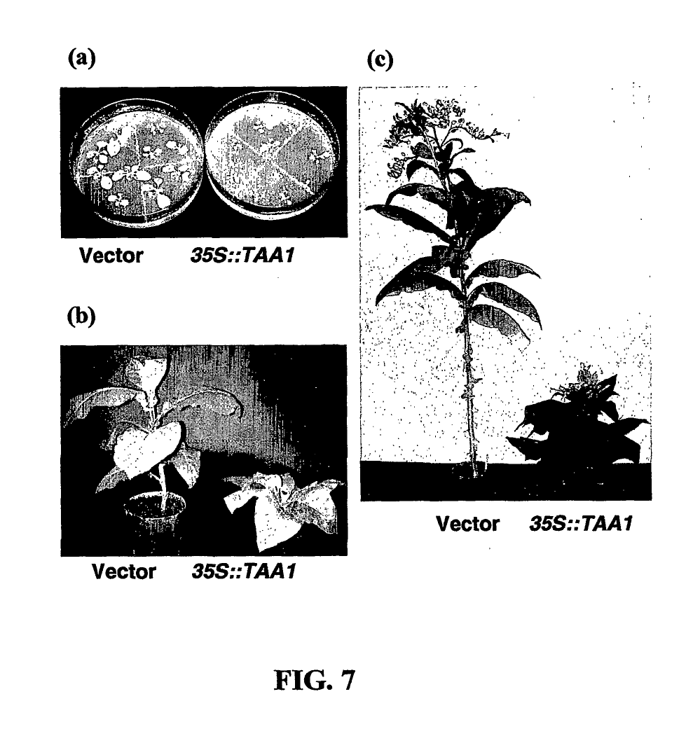

Unexpectedly, the overexpression of the TAA1a gene in tobacco under the control of a 35S promoter results in significant changes to the phenotype of the corresponding transgenic plants (FIG. 7). In this regard, the modified transgenic plants are significantly smaller, with shorter intemodes, and delayed flowering. The expression of fatty acyl Co-A reductase in these plants therefore gives rise to considerable developmental alterations in the plant. This provides evidence that changes in lipid metabolism via altered expression of TAA1 genes can generate desirable changes to plant phenotype. Specifically, the reduction in internode length and reduction in overall size of the plants will render the plants less susceptible to wind damage. Moreover, the reduction in size of the plants may permit the generation of dwarf plant specifies for horticultural purposes, and plants with increased wind resistance. The delay in flowering may also be a desirable attribute for certain horticultural situations.

### EXAMPLE 8

### Accumulation of Fatty Alcohols from TAA1 Expression in E. coli

To further verify TAA1's alcohol-forming ability, a bacterial expression system was employed. Previously this approach had been originally used to determine the enzymatic activity of the FAR isolated from jojoba (Metz et al., 2000). The neutral components from bacterial cells with the control plasmid or with the plasmid containing the coding region of the TAA1a cDNA under the control of the T7 promoter were subjected to GC analysis. In the control bacterial cells, there was one major peak observed. The nature of this compound was not clear. In the bacterial cells expressing TAA1a, two additional major peaks and one additional minor peak were detected (FIG. 6b). Detention times of these three peaks were found to be identical to those of three authentic fatty alcohol standards (C14, myristi alcohol; C16:0, hexadecyl alcohol; and C18:1, oleyl alcohol). The identity of the two major peaks (C16 and C18:1) was confirmed by MS-GC while the C14 fatty alcohol was not due to its very low concentration. These results suggest that TAA1 is a wheat FAR.

### EXAMPLE 9

### PROMOTER STUDIES—The Wheat TAA1b Promoter Retains its Spatial and Temporal Expression Specificity in a Distant Monocot and also in a Dicot

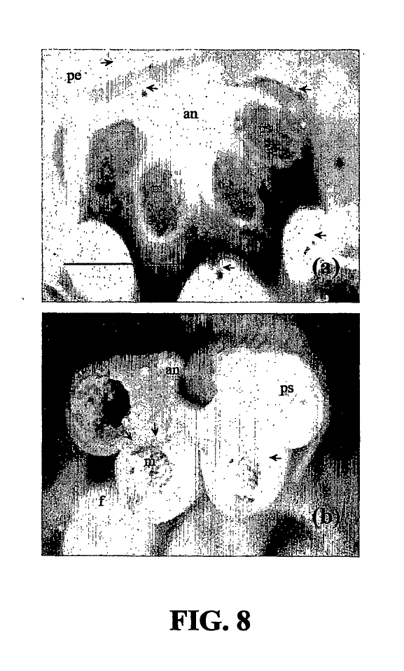

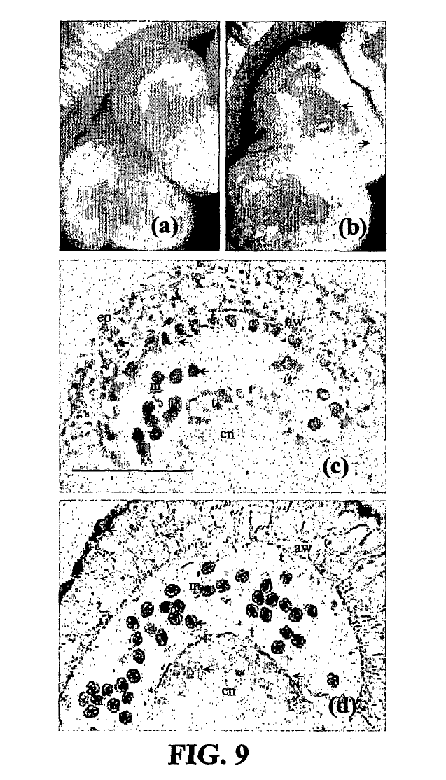

A 1.6-Kb genomic segment upstream of the predicted start codon of the TAA1b was isolated by genome walking (and given GenBank accession no: AJ488930). Particle-bombardment of daylily, a distant monocot in the phylogeny of wheat, with a construct containing GUS ORF immediately 3′ to this fragment elaborated β glucuronidase, as shown by histochemical staining, only in the anther tapetum and microspores (FIG. 8); none of the other parts such as leaves, stems, and the anther epidermis and connective tissues showed GUS expression. Furthermore, when anthers of various stages of development were bombarded, only those past the tetrad formation showed GUS expression, and this result was consistent with the observations in wheat. In contrast, a CaMV 35S-GUS construct gave expression in all tissues examined. Thus, the wheat promoter is likely to be useful to manipulate male gametophyte development in other important monocot crops. The wheat TAA1 promoter was also found to be functional in the anthers of stably transformed tobacco (FIG. 9). GUS expression was absent in other floral parts, roots and stems. In contrast, and as expected, the CaMV 35S-GUS plants showed expression in all these tissues, and an example of floral tissue is shown for comparison (FIG. 9d). While GUS activity was evident in the epidermis, connective tissues, the tapetum, microspores, and other tissues, the expression in the tapetum was not as strong as in the case of the TAA1b-GUS plant. Interestingly, the heterologous promoter maintained its developmental specificity in transgenic tobacco. GUS staining was not detectable in very young anthers at the time of tetrad formation (FIG. 9a). According to Koltunow et al. (1990), this would be flower buds <12 mm. In flower buds of 14 to 24 mm (Stages 2 to 6), GUS activity was detected in the tapetum, and in buds >15 mm (Stage 4), a strong GUS staining was also seen in microspores (FIG. 9b and c). Collectively, these results show cross-functionality of any cis elements of the TAA1 promoter with trans-acting factors in daylily and tobacco.

Experimental Procedures

Plant materials, DNA and RNA isolation

Hexaploid spring wheat (Triticum aestivum L. cv. Karma, genomic complement AABBDD), tetraploid wheat (Triticum turgidum L. cv. Sceptre, genomic complement AABB), and two diploid wheat species (Triticum urartu, ssp. Nigrum, genomic complement AA; Aegilops squarrosa, ssp. Tauschii, genomic complement DD) were grown in an open filed or in a greenhouse under standard conditions. Five-week-old seedlings were used for DNA isolation and for leaf, root and stem RNA isolation. Anther, ovary, glume and pilea tissues were collected 1-3 days prior to anthesis for RNA purification. DNA extraction was carried out following the published protocol (Wang et al., 1998). Total RNA was extracted using Trizol™ reagent (Life Technologies/Gibco-BRL, Burlington, Ontario) following the supplier's recommendations.

RT-PCR, 5′- and 3 ′-RACE, and other RNA and DNA technologies

The nucleotide acid-related enzymes used were from Life Technologies (Burlington, Canada) except otherwise stated. Five μg of total RNA derived from various tissues was used to synthesize the first strand cDNA by reverse transcriptase using primers OL2707 (5′GACTACGTCGTCCAAGGCCG3′-SEQ ID NO: 10) and OL2708 (5′GTCGAACTGCTTGAGCAG CGC3′-SEQ ID NO: 11). The PCR reactions were carried out with a Techne Genius DNA thermal cycler (Duxford, Cambridge, UK) under the following conditions: 94° C. for 1 min, 43° C. for 1 min, 72° C. for 2 min, 35 cycles, followed by 10 min's incubation at 72° C. The amplified products were subjected to DNA sequencing. Based on obtained sequences, primers OL2881 (5′GCAGAACCTGACATACTTC3′-SEQ ID NO: 12) and OL2885 (5′GAGGCGGTACCT GAGCAT3′-SEQ ID NO: 13) were designed for RT-PCR detection and genomic DNA amplification.

Antisense primers OL2884 (5′TTCGCATAGCCGATCACG3′-SEQ ID NO: 14) and OL2883 (AATGCCGGCCCTGGTAAG3′-SEQ ID NO: 15) and sense primers OL2880 (5′CAGGTGGCCAAA CACATA SEQ ID NO: 16 and OL2881 (5′GCAGAACCTGACATACTTC-SEQ ID NO: 12) were designed for 5′- and 3′-RACE which were conducted using 5′ and 3′ RACE kits (Life Technologies, Burlington, Ontario) following the manufacturer's protocol. The resulting cDNA fragments were either directly subjected to DNA sequencing or cloned into a T/A vector using the Original TA Cloning Kit™ (Invitrogen, Carlsbad, Calif.) for sequencing. RNA and DNA gel blot analyses were performed essentially as described (Nair et al., 2000). The predicted coding region of TAA1a was used as a probe.

cDNA library construction and screening

Wheat anther poly(A)⁺ RNA was isolated using an mRNA kit (Clontech, Palo Alto, Calif.). Approximately 5 μg poly(A)⁺ RNA was used for cDNA library construction using a ZAP cDNA Gigapack III Gold Cloning Kit™ according to the supplier's instructions (Stratagene, La Jolla, Calif.). The ligated vector was packaged into phage particles using Gigapack III gold packaging extracts (Stratagene, La Jolla, Calif.). A total of 2.2×10⁷ primary pfu were obtained. Library screening was conducted using the 5′ RACE PCR cDNA fragment as a probe to hybridize phage plaques containing approximately 250,000 recombinant clones. The positive plaques were isolated and the phagemids were excised in vivo from the Uni-ZAP XR™ vector using the ExAssist/SOLR™ system (Stratagene, La Jolla, Calif.). The inserted cDNA sequences in the purified phagemids were determined by DNA sequencing.

Production ofpolyclonal antibodies against TAA1

The entire coding region of TAA1a was directionally cloned in-frame into the BamH1-EcoRI sites of plasmid pRSET A (Invitrogen, Carlsbad, Calif.) to make plasmid pTAA238. Fusion protein was expressed in E. coli strain BL21(DE3)pLysS (Invitrogen). The TAA1 fusion protein was purified and injected into rabbits following the procedures (Wang et al., 1999). Polyclonal antibodies were harvested and purified as described (Wang et al., 1999).

In situ RNA hybridization and immuno-cytolocalization

Plant materials were infiltrated overnight at 4° C. in 4% paraformaldehyde (PFA) with a 100 mM phosphate buffer pH 7.2. The fixed material were dehydrated in a graded ethanol series and then embedded in paraffin (Paraplast plus x-tra). Sections were cut at 8 μm thickness and mounted on glass slides (Superfrost™ plus, Fisher Scientific, Nepean, Ontario). For in situ RNA hybridization, probes were prepared using MAXIscript™ in vitro transcription kit and BrightStar™ P soralen-Biotin nonisotopic labeling kit (Ambion, Austin, Tex.) according to manufacture's protocols. A DNA fragment of 550 bp of the TAA1a cDNA starting from the predicted start codon was directionally cloned into pBluescript™ II KS⁺ phagemid vector (Stratagene, La Jolla, Calif.) at the BamHI-XhoI sites to produce plasmid pTAA253. The antisense transcripts synthesized in vitro by T3 polymerase using XbaI-linearlized plasmid pTAA253 as a template were used to detect the TAA1 mRNA. The partial TAA1 sense transcripts generated by T7 polymerase using plasmid XhoI-linearized plasmid pTAA253 as a template were served as a control. In situ hybridization was carried out essentially following the instructions of the mRNA locator-HybM kit (Ambion).

For in situ immunological detection, slides mounted with fixed sections of wheat florets are incubated in the blocking solution containing 1:1000 TAA1a immune or pre-immune serum for overnight at 4° C. Visualization of immuno-reaction was as described (Cho and Kende, 1998).

Vector construction and genetic transformation

Plasmid pRD400 (Datla et al., 1992) was modified by flipping-over the region containing the polylinker and the NPT II gene cassette to generate a binary transformation vector pAMW281. Two pieces of DNA fragments including a 2.4 kb fragment containing a CaMV 35S promoter and a uidA gene from plasmid pRD410 (Datla et al., 1992) digested with HindIII and EcoRI, and a 0.7 kb fragment containing a CaMV 35S terminator from plasmid pHS724 restricted with EcoRI and KpnI (Huang et al., 2000) were co-ligated into the backbone of pAMW281 digested with HindIII and KpnI to produce plasmid pAMW287. A 1.4 kb napin promoter obtained from digestion of plasmid pJOY43 with HindIII-BamHI (Nair et al., 2000) and the 1.4 kb TAA1a entire coding region resulting from plasmid pTAA238 restricted with BamHI and EcoRI were co-ligated into the HindIII-EcoRI sites of plasmid pAMW287. The resulting plasmid pAMW 458 consisted of the Napin promoter, the TAA1a coding region and the 35S transcription terminator.

Agrobacterium-mediated transformation was employed for production of tobacco (Nicotiana tabacuni cv Xanthi) transgenic plants using published protocols (Huang et al., 2000). The presence of foreign genes in independently derived kanamycin-resistant cell lines was confirmed by PCR and Southern blot analyses, according to standard techniques.

Gas chromatography (GC) analysis

For plant GC analysis, mature seeds were harvested from TAA1 transgenic plants, non-transgenic wild-type control plants and control transgenic plants. Seed samples were ground and saponified with 10% potassium hydroxide dissolved in methanol with 1% water, incubated at 80° C. for 2 hours. The mixture was then extracted twice with hexane and subjected to GS analyses for total fatty alcohol and fatty acid contents and compositions as described by Katavic et al. (1995). Analysis was done on a 30 M DB-5 column starting at 250° C. to 300° C. at 5° C./min. The identity of the fatty alcohol peaks was based on retention times of authentic fatty alcohol standards and confirmed by GC-MS. The relative amount of fatty alcohols was calculated on the basis of fresh weight of the seeds and normalized according to internal contents of β-sitosterol extracted in the same procedure.

For E. coli GC analyses, 200 ml bacterial cells with appropriate plasmids were grown to OD value of 0.5 at 30° C. After addition of IPTG (0.2 mM), the culture was allowed to grow for 3 hr. The bacterial cells were harvested. Subsequent extraction and GC analysis were essentially as above. Qualification of fatty alcohols was based on flame ionization detector peak areas, which were converted to mass units by comparison with an internal standard which was added before the extraction.

TAA1 promoter isolation and analysis

The upstream regulation region of TAA1b was isolated from the hexaploid spring wheat cultivar Karma (genetic complements: AABBDD) using a Universal GenomeWalker™ Kit (Clontech, Palo Alto, Calif.). The resulting 1.7 kb DNA fragment was cloned into a T/A vector (Original TA Cloning Kit, Invitrogen, Carlsbad, Calif.) for further analysis.

Plasmid pRD400 (Datla et al., 1992) was modified by flipping-over the region containing the polylinker and the NPT II gene cassette to generate a binary transformation vector pAMW281. Two pieces of DNA fragments including a 2.4 kb fragment containing a CaMV 35S promoter and a uidA gene from plasmid pRD410 (Datla et al., 1992) digested with HindIII and EcoRI, and a 0.7 kb fragment containing a CaMV 35S terminator from plasmid pHS724 restricted with EcoRI and KpnI (Huang et al., 2000) were co-ligated into the backbone of pAMW281 digested with HindIII and KpnI to produce plasmid pAMW287, consisting of the 35S-GUS-PolyA cassette. Plasmid pAMW445 containing TAA1b promoter-GUS-PolyA was obtained by cloning the isolated 1.5 kb TAA1b promoter into the HindIII-BamHI sites of plasmid pAMW287. Agrobacterium-mediated transformation was employed for production of tobacco (Nicotiana tabacum cv Xanthi) transgenic plants using published protocols (Huang et al., 2000). Presence of foreign genes in independently derived kanamycin-resistant cell lines was confirmed by PCR and Southern blot analyses.

For transient expression analysis, microprojectiles coated with 35S/GUS or TAA1 promoter/GUS chimeric genes were bombarded into the transverse sections of flowers of a monocotyledonous plant species, daylily (Hemerocallis lilioasphodelus) essentially as described (Chen et al., 1998).

To analyze GUS expression in transgenic plants, the flower buds were collected from the primary transgenic tobacco plants (F₀). Anthers were cut transversely and incubated in a GUS-assay buffer (0.1 M phosphate buffer pH7.0, 2 mM K₃[Fe(CN)₆], 2 mM K₄[Fe(CN)₆], 1 mM EDTA. 0.1% Triton) with 1 mM X-Gluc (5-bromo-4-chloro-3-indoyl-β-D-glucuronide) overnight at 37° C. After incubation, the anthers were observed under a microscope. Typical anthers were embedded in paraffin and then sectioned in 6 μm thickness for the further observation. The histochemical assay on the daylily flowers was performed 24 h post bombardment essentially as described (Wang et al., 1998).

Whilst the present invention has been described with particular reference to specific examples and techniques, the invention is not intended to be limited in this regard, and numerous peptide and nucleotide sequences, constructs, transformed organisms, methods, and products that are not directly described herein are intended to be encompassed within the scope of the present invention.

### References

Aarts M G M, Dirkse W G, Stiekma W J, Pereira A (1993) Transposon tagging of a male sterility gene in Arabidopsis. Nature 353: 715-717

Aarts M G M, Hodge R, Kalantidis K, Florack D, Wilson Z A, Mulligan B, Stiekema W J, Scott R, Pereira A (1997) The Arabidopsis MALE STERILITY 2 protein shares similarity with reductases in elongation condensation complexes. Plant Journal 12: 615-623

Aarts M G M, Keijzer C J, Stiekema W J, Pereira A (1995) Molecular characterization of the CER1 gene of Arabidopsis involved in epicuticular wax biosynthesis and pollen fertility. Plant Cell 7: 2115-2127

Aguirre P J, Smith A G (1993) Molecular characterization of a gene encoding a cycteine-rich protein preferentially expressed in anthers of Lycopersicon esculentum. Plant Molecular Biology 23: 477-487

Altschul S F, Madden T L, Schäffer A, Zhang J, Zhang Z, Miller W, Lipman D J (1997) Gapped BLAST and PSI-BLAST: a new generation of protein database search programs. Nucleic Acids Research 25: 3389-3402

Bih F Y, Wu S S H, Ratnayake C, Walling L L, Nothnagel E A, Huang A H C (1999) The predominant protein on the surface of maize pollen is an endoxylanase synthsized by a tapetum mRNA with a long 5′ leader. The Journal of Biological Chemistry 274: 22884-22894

Brown S M, Crouch M L (1990) Characterization of a gene family abundantly expressed in Oenothera organensis pollen that shows sequence similarity to polygalacturonase. Plant Cell 2: 263-274

Chen L, Zhang S, Beachy R N, Fauquet C M (1998) A protocol for consistent, large-scale production of fertile transgenic rice plants. Plant Cell Report 18: 25-31. Plant Cell Report 18: 25-31

Cho H-T, Kende H (1998) Tissue localization of expression in deep water rice. Plant Journal 15: 805-812

Datla R S S, Hammerlindl J K, Panchuk B, Pelcher L E, Keller W (1992) Modified binary plant transformation vectors with the wild-type gene encoding NPTII. Gene 122: 383-384

Fuerstenberg S, Bucciaglia P A, Smith A G (2000) Molecular characterization of an anther-specific geen from tobacco shows sequence similarity to a tapetum-specific gene from tomato. Sex Plan Reprod 12: 250-252

Futamura N, Mori H, Kouchi H, Shinohara K (2000) Male flower-specific expression of gene for polygalacturonase, pectin methylesterase and β-1,3-glucanase in a dioecious willow (Salix gilgiana Seemen). Plant Cell Physiology 41: 16-26

Goldberg R B, Beals T P, Sanders P M (1993) Anther development: basic principles and practical applications. Plant Cell 5: 1217-1229

Hernindez-Pinzon I, Ross J H E, Barnes K A, Damant A P, Murphy D J (1999) Composition and role of tapetal lipid bodies in the biogenesis of the pollen coat of Brassica napus. Planta 208: 588-598

Hird D L, Worral D, Hodge R, Smartt S, Wyatt P, Scott R (1993) The anther-specific protein encoded by the Brassica napus and Arabidopsis thaliana a6 gene displays similarity to β-13-γλυχανασεσ. Plant Journal 4: 1023-1033

Hodge R, Paul W, Draper J, Scott R (1992) Cold-plaque screening: a simple technique for the isolation of low abundance, differentially expressed transcripts from conventional cDNA libraries. Plant Journal 2: 257-267

Huang J, Hirji R, Adam L, Rozwadowski K L, Hammerlindl J K, Keller W A, Selvaraj G (2000) Genetic engineering of glycinebetaine production toward enhancing stress tolerance in plants: metabolic limitations. Plant Physiology 122: 747-756

Jeon J S, Chung Y Y, Lee S, Yi G H, Oh B H, An G (1999) Isolation and characterization of an anther-specific gene, RA8, from rice (Oryza sativa L.). Plant Molecular Biology 39: 35-44

Kandasamy M K, McKinney E C, Meagher R B (1999) The late pollen-specific actins in angiosperms. The Plant Journal 18: 681-691

Katavic, V., Reed, D. W., Taylor, D. C., Giblin, E. M., Barton, D. L., Zou, J.-T., MacKenzie, S. L., Covello, P. S. & Kunst, L. (1995). Alternation of seed fatty acid composition by an ethyl methanesulfonate-induced mutation in Arabidopsis thaliana affecting diacylglycerol acyltransferase activity. Plant Physiology 108, 399-409.

Kolattukudy P E (1970) Reduction of fatty acids to alcohols by cell-free preparations of Euglena gracilis. Biochemistry 9: 1095-1102

Koltunow A M, Truettner J, Cox K H, Wallroth M, Goldberg R B (1990) Different temporal and spatial gene expression patterns occur during anther development. Plant Cell 2: 1201-1224

Lopez I, Anthony R G, Maciver S K, Jiang C-J, Khan S, Weeds A G, Hussey P J (1996) Pollen specific expression of maize genes encoding actin deploymerizing factor-like proteins. Proc Natl Acad Sci USA 93: 7415-7420

Mariani C, de Beuckeleer M, Truettner J, Leemans J, Goldberg R B (1990) Induction of male sterility in plants by a chimaeric ribonuclease gene. Nature 347: 737-741

Mascarenhas J P (1975) The biochemistry of angiosperm pollen development. Bot Rev 41: 259-314

May C E, Appels R (1987) Wheat worldwide in “Wheat and Wheat Improvement” (Ed. E. G. Heyne) pp 165-198. Agronomy No. 13. American Society of Agronomy, Inc Madison, Wis., USA

Metz J G, Pollard M R, Anderson L, Hayes T R, Lassner M W (2000) Purification of a jojoba embryo fatty acyl-coenzyme A reductase and expression of its cDNA in high erucic acid rapeseed. Plant Physiology 122: 635-644

Millar A A, Clemens S, Zachgo S, Giblin E M, Taylor D C, Kunst L (1999) CUT1, an Arabidopsis gene required for cuticular wax biosynthesis and pollen fertility, encodes a very-long-chain fatty acid condensing enzyme. Plant Cell 11: 825-838

Nakamura T, Yamamori M, Hirano H, Hidaka S, Nagamine T (1995) Production of waxy (amylose-free) wheats. Molecular General Genetics 248: 253-259

Nair, R. B., Joy IV, R. W., Kurylo, E., Shi, X., Schnaider, J., Datla, R. S. S., Keller, W. A. & Selvaraj, G. (2000). Identification of a CYP84 family of cytochrome P450-depdendent mono-oxygenase genes in Brassica napus and perturbation of their expression for engineering sinapine reduction in the seeds. Plant Physiology 123, 1623-1634.

Pearson W R, Lipman D J (1988) Improved tools for biological sequence comparison. Proc Natl Acad Sci USA 85: 2444-2448

Pickett A A, Galwey N W (1997) A further evaluation of hybrid wheat. Plant Varieties and Seeds 10: 15-32

Piffanelli P, Ross J H E, Murphy D J (1998) Biogenesis and function of the lipidic structures of pollen grains. Sex Plan Reprod 11: 65-80

Preuss D, Lemieux B, Yen G, Davis R W (1993) A conditional sterile mutation eliminates surface components from Arabidopsis pollen and disrupts cell signaling during fertilization. Genes & Development 7: 974-985

Rafnar T, Griffith I J, Kuo M, Bond J F, Rogers B L, Klapper D G (1991) Cloning of Amb a I (antigen E), the major allergen family of short ragweed pollen. Journal of Biological Chemistry 266: 1229-1236

Rubinelli P, Hu Y, Ma H (1998) Identification, sequencing analysis and expression studies of novel anther-specific genes of Arabidopsis thaliana. Plant Molecular Biology 37: 607-619

Scott R J Pollen exine—the sporopollenin enigma and the physics of pattern. In: Scott R J, Stead A D (eds) Molecular and cellular aspects of plant reproduction, pp. 49-81. Cambridge University Press, Cambridge (1994).

Shivanna K R, Cresti M, Ciampolini F (1997) Pollen development and pollen-pistil interaction. In: Shivanna KR, Sawhney VK (eds) Pollen biotechnology for crop production and improvement. Cambridge University Press, Cambridge, United Kingdom.

Spencer D F, Schnare M N, Coulthart M B, Gray M W (1992) Sequence and organization of a 7.2 kb region of wheat mitochondrial DNA containing the large subunit (26S) rRNA gene. Plant Molecular Biology 20: 347-352

Ting J T L, Wu S S H, Ratnayake C, Huang A H C (1998) Constituents of the tapetosomes and elaioplasts in Brassica campestris tapetum and their degradation and retention during microsporogenesis. Plant Journal 16: 541-551

Vioque J, Kolattukudy P E (1997) Resolution and purification of an aldehyde-generating and an alcohol-generating fatty acyl-CoA reductase from pea leaves (Pisum sativum L.). Arch Biochem Biophys 340: 64-72

Walden A R, Walter C, Gardner R C (1999) Genes expressed in Pinus radiata male cones include homologs to anther-specific and pathogenesis response genes. Plant Physiology 121: 1103-1116

Wang A, Carrier K, Chisholm J, Wieczorek A, Huguenot C, Sanfacon H (1999) Porteolytic processing of tomato ringspot nepovirus 3C-like protease precursors: definition of the domains for the VPg, protease and putative RNA-depedent RNA polymerase. J Gen Virol 80: 799-809

Wang A, Fan H, Singsit C, Ozias-Akins P (1998) Transformation of peanut with a soybean vspB promoter-uidA chimeric gene. I. Optimization of a transformation system and analysis of GUS expression in primary transgenic tissues and plants. Physiologia Plantarum 102: 38-48

Zou J-T, Zhan X-Y, Wu H-M, Wang H, Cheung A Y (1994) Characterization of a rice pollen-specific gene and its expression. American Journal of Botany 81: 552-561

1. An isolated and purified nucleotide sequence, characterized in that the nucleotide sequence is endogenously expressed in wheat anthers, and encodes a peptide having fatty acyl Co-A reductase (FAR) activity.

2. An isolated and purified nucleotide sequence, characterized in that the nucleotide sequence is selected from:
(a) a TAA1 gene, or a part thereof, or a complement thereof; and
(b) a nucleotide sequence encoding a peptide having at least 50% identity to a peptide encoded by a TAA1 gene, or a part thereof, or a complement thereof,
said nucleotide sequence encoding a protein or a part thereof, that alters lipid metabolism in a transgenic plant exogenously expressing said nucleotide sequence.

3. An isolated and purified nucleotide sequence according to claim 2, characterized in that the nucleotide sequence is selected from:
(a) SEQ ID NO: 1, 3, or 5, or a part thereof, or a complement thereof; and
(b) a nucleotide sequence encoding a peptide having at least 50% identity to a peptide encoded by a SEQ ID NO: 1, 3, or 5, or a part thereof, or a complement thereof;
said nucleotide sequence encoding a protein or a part thereof, that alters lipid metabolism in a transgenic plant exogenously expressing said nucleotide sequence.

4. An isolated and purified nucleotide sequence according to claim 2, characterized in that the nucleotide sequence is selected from:
(a) SEQ ID NO: 1, 3, or 5, or a part thereof, or a complement thereof; and
(b) a nucleotide sequence encoding a peptide having at least 70% identity to a peptide encoded by a SEQ ID NO: 1, 3, or 5, or a part thereof, or a complement thereof;
said nucleotide sequence encoding a protein or a part thereof, that alters lipid metabolism in a transgenic plant exogenously expressing said nucleotide sequence.

5. An isolated and purified nucleotide sequence according to claim 2, characterized in that the nucleotide sequence is selected from:
(a) SEQ ID NO:1, 3, or 5, or a part thereof, or a complement thereof; and
(b) a nucleotide sequence encoding a peptide having at least 90% identity to a peptide encoded by a SEQ ID NO: 1, 3, or 5, or a part thereof, or a complement thereof;
said nucleotide sequence encoding a protein or a part thereof, that alters lipid metabolism in a transgenic plant exogenously expressing said nucleotide sequence.

6. An isolated and purified nucleotide sequence according to claim 2, characterized in that the nucleotide sequence is selected from:
(a) SEQ ID NO: 1, 3, or 5, or a part thereof, or a complement thereof; and
(b) a nucleotide sequence encoding a peptide having at least 95% identity to a peptide encoded by a SEQ ID NO: 1, 3, or 5, or a part thereof, or a complement thereof;
said nucleotide sequence encoding a protein or a part thereof, that alters lipid metabolism in a transgenic plant exogenously expressing said nucleotide sequence.

7. An isolated and purified nucleotide sequence according to claim 2, characterized in that the nucleotide sequence is selected from:
(a) SEQ ID NO: 1, 3, or 5, or a part thereof, or a complement thereof; and
(b) a nucleotide sequence encoding a peptide having at least 99% identity to a peptide encoded by a SEQ ID NO: 1, 3, or 5, or a part thereof, or a complement thereof;
said nucleotide sequence encoding a protein or a part thereof, that alters lipid metabolism in a transgenic plant exogenously expressing said nucleotide sequence.

8. An isolated and purified nucleotide sequence according to claim 3, characterized in that the nucleotide sequence is selected from:
(a) SEQ ID NO: 1, 3, or 5, or a part thereof, or a complement thereof; and
(b) a nucleotide sequence that can hybridize to SEQ ID NO: 1, 3, or 5 under stringent hybridization conditions.

9. An expression cassette, characterized in that the expression cassette comprises the nucleotide sequence according to claim 2, operably linked to a promoter.

10. The expression cassette according to claim 9, characterized in that the nucleotide sequence is oriented in an antisense direction, or in both a sense and an antisense direction to produce double stranded RNA, relative relative to said promoter.

11. A construct, characterized in that the construct comprises the nucleotide sequence according to claim 2.

12. The construct according to claim 11, characterized in that the construct includes a promoter, said nucleotide sequence operatively linked to said promoter.

13. A transgenic plant, characterized in that the transgenic plant comprises the construct according to claim 12, said nucleotide sequence expressed from said promoter thereby altering a lipid metabolism of said transgenic plant.

14. The transgenic plant according to claim 13, characterized in that said plant comprises an increased concentration of fatty alcohols per weight of plant material relative to an unmodified plant.

15. The transgenic plant according to claim 13, characterized in that said plant comprises a decreased concentration of fatty alcohols per weight of plant material relative to an unmodified plant.

16. The transgenic plant according to claim 13, characterized in that the transgenic plant comprises pollen grains or seeds having an altered fatty alcohol content relative to pollen grains or seeds of an unmodified plant.

17. The transgenic plant according to claim 13, characterized in that said plant is a species of a woody plant, a non-woody plant, or a grass.

18. The transgenic plant according to claim 13, characterized in that said plant is selected from the group consisting of crucifer crops, tobacco, wheat, corn, sugar cane, apple, tomato, and berries.

19. The transgenic plant according to claim 13, characterized in that said construct comprises an organ-specific promoter to direct organ-specific expression of said nucleotide sequence in said transgenic plant.

20. The transgenic plant according to claim 13, characterized in that said transgenic plant exhibits modified characteristics compared to an unmodified plant, said modified characteristics selected from the group consisting of:
increased pest resistance;
male sterility;
reduced height;
reduced internode spacing;
increased resistance to wind damage;
reduced growth rate;
altered cross-pollination specification;
increased fruit or nut aesthetic appeal;
increased fruit or nut shelf-life;
delayed vegetative development; and
delayed propagative development.

21. The transgenic plant according to claim 13, characterized in that said nucleotide sequence is oriented for antisense expression from said constructs said transgenic plant exhibiting reduced levels of fatty acyl Co-A reductase compared to an unmodified plant.

22. A plant extract, characterized in that said plant extract is derived from a transgenic plant according to claim 13 for use in the production of a nutritional, cosmetic or pharmaceutical agent.

23. An isolated and purified nucleotide sequence according to claim 2, characterized in that the nucleotide sequence is selected from:
(a) SEQ ID NO: 7, 8, or 9 or a complement thereof; and
(b) a nucleotide sequence that can hybridize to SEQ ID NO: 7, 8, or 9 or a complement thereof under stringent hybridization conditions.

24. The isolated and purified nucleotide sequence according to claim 23, characterized in that said nucleotide sequence is for use as a hybridization probe, PCR primer or DNA sequencing primer.

25. A promoter sequence, characterized in that said promoter sequence regulates gene expression of anthers and is isolated from a genomic DNA library by chromosome walking, using as a probe the isolated and purified nucleotide sequence according to claim 24.

26. A promoter sequence according to claim 25, characterized in that said promoter sequence comprises a region of about 1.6 kb upstream from a start codon of SEQ ID NO: 8.

27. A construct, characterized in that said construct comprises a promoter sequence according to claim 25, operably linked to a nucleotide sequence having an open reading frame, or a part thereof, or a complement thereof.

28. A transgenic plant, characterized in that said transgenic plant is transformed with a construct according to claim 27, said promoter inducing expression of said open-reading frame, or a part thereof, or a complement thereof, in at least one anther of said transgenic plant.

29. The transgenic plant according to claim 28, characterized in that said open-reading frame encodes an anther or pollen inactivating gene, and expression of said open-reading frame induces male sterility of said transgenic plant.

30. The transgenic plant according to claim 28, characterized in that said open-reading frame encodes a transposase, and expression of said open-reading frame induces an increased rate of genomic DNA rearrangement in anther or pollen cells of said transgenic plant.

31. The transgenic plant according to claim 28, characterized in that said open-reading frame encodes a peptide suitable for use as a nutritional or pharmaceutical agent, said peptide expressed in anthers or pollen of said transgenic plant.

32. The transgenic plant according to claim 28, characterized in that said open-reading frame encodes a peptide required for the production of a nutritional or pharmaceutical agent.

33. The transgenic plant according to claim 28, characterized in that said open-reading frame encodes a protein that inhibits the production and/or accumulation of an unwanted substance selected from the group consisting of a toxin, and an allergen.

34. The transgenic plant according to claim 28, characterized in that said open-reading frame encodes a peptide for altering a cross-pollination specification of said transgenic plant.

35. The transgenic plant according to claim 28, characterized in that said open reading frame is oriented for antisense expression within said construct, thereby inducing antisense repression of an endogenous gene expression within anthers or pollen of said transgenic plant.

36. The transgenic plant according to claim 32, characterized in that said nutritional agent is Octacosanol.

37. A plant extract, characterized in that the plant extract is derived from a transgenic plant according to claim 28.

38. The transgenic plant according to claim 13, characterized in that the plant comprises fruit, and the plant exhibits increased levels of fatty alcohols in said fruit to preserve and/or provide an enhanced appearance of said fruit.

39. Use for Octacosanol derived from the transgenic plant according to claim 36, characterized in that said Octacosanol is for use as a nutritional supplement.

40. Use for fatty alcohols derived from the transgenic plant according to claim 13, characterized in that said use is selected from:
use as a wax;
use as a cleaning agent;
use as a cosmetic agent;
use as a dermatological agent;
use as a pharmaceutical agent;
use as a nutritional agent; and
use as a coating agent.

41. An isolated and purified peptide, characterized in that said isolated and purified peptide is selected from the group consisting of:
(a) SEQ ID NO: 2, 4, or 6, or a part thereof; and
(b) a peptide having at least 50% identity to a peptide encoded by a SEQ ID NO: 2, 4, or 6, or a part thereof;
said peptide or a part thereof altering lipid metabolism in a transgenic plant when generated by exogenous gene expression in said transgenic plant.

42. An isolated and purified peptide according to claim 41, characterized in that said isolated and purified peptide is selected from the group consisting of:
(a) SEQ ID NO: 2, 4, or 6, or a part thereof; and
(b) a peptide having at least 70% identity to a peptide encoded by a SEQ ID NO: 2, 4, or 6, or a part thereof;
said peptide or a part thereof altering lipid metabolism in a transgenic plant when generated by exogenous gene expression in said transgenic plant.

43. An isolated and purified peptide according to claim 41, characterized in that said isolated and purified peptide is selected from the group consisting of:
(a) SEQ ID NO: 2, 4, or 6, or a part thereof; and
(b) a peptide having at least 90% identity to a peptide encoded by a SEQ ID NO: 2, 4, or 6, or a part thereof;
said peptide or a part thereof altering lipid metabolism in a transgenic plant when generated by exogenous gene expression in said transgenic plant.

44. An isolated and purified peptide according to claim 41, characterized in that said isolated and purified peptide is selected from the group consisting of:
(a) SEQ ID NO: 2, 4, or 6, or a part thereof; and
(b) a peptide having at least 95% identity to a peptide encoded by a SEQ ID NO: 2, 4, or 6, or a part thereof;
said peptide or a part thereof altering lipid metabolism in a transgenic plant when generated by exogenous gene expression in said transgenic plant.

45. An isolated and purified peptide according to claim 41, characterized in that said isolated and purified peptide is selected from the group consisting of:
(a) SEQ ID NO: 2, 4, or 6, or a part thereof; and
(b) a peptide having at least 99% identity to a peptide encoded by a SEQ ID NO: 2, 4, or 6, or a part thereof;
said peptide or a part thereof altering lipid metabolism in a transgenic plant when generated by exogenous gene expression in said transgenic plant.

46. A pharmaceutical composition, characterized in that the pharmaceutical composition comprises a fatty alcohol derived from the transgenic plant according to claim 13 or claim 28, together with an excipient or carrier.

47. A pharmaceutical composition, characterized in that the pharmaceutical composition comprises the plant extract according to claim 22 or claim 37, together with an excipient or carrier.

48. A nutritional composition, characterized in that the nutritional composition comprises a fatty alcohol derived from the transgenic plant according to claim 13 or claim 28 together with an excipient or carrier.

49. A nutritional composition, characterized in that the nutritional composition comprises the plant extract according to claim 22 or claim 37 together with an excipient or carrier.

50. A method of treating or preventing a medical condition, characterized in that the method comprises administration a pharmaceutical composition according to claim 46 or claim 47.

51. A method of providing a dietary supplement, characterized in that the method comprises administration of a nutritional composition according to claim 48 or claim 49.

52. A method for the production and isolation of fatty alcohols, characterized in that the method comprises the steps of:
transforming an organism with a construct according to claim 11;
growing or propagating said organism containing said construct; and
extracting said fatty alcohols from said organism.

53. A method according to claim 52, characterized in that said organism is an E.coli bacterium.

54. A method according to claim 52, characterized in that said organism is a plant, or a plant embryo.

55. A method according to claim 52, characterized in that said organism is a tobacco plant, or a tobacco plant embryo.

56. Isolated and purified fatty alcohols, characterized in that said fatty alcohols are obtained by a method according to claim 52.

57. A method of inducing dwarfism in a plant, characterized in that the method comprises the steps of:
transforming a plant cell, plant embryo or plant with a construct according to claim 11;
growing or propagating said plant cell, plant embryo, or plant, thereby generating a plant expressing a DNA sequence encoded by said construct, said plant having a reduced size compared to an unmodified plant.

58. A plant characterized in that the plant is generated by the method according to claim 57.

59. The plant according to claim 58, characterized in that said plant exhibits an increased resistance to wind damage compared to an unmodified plant.


SEQUENCE LISTING (16 sequences)

SEQ ID NO: 1 - DNA, 1919 bp, Triticum aestivum
  CDS (70)..(1593)
ctcctctctctctctctctctctctctctctcgcacgcacgcacgcacgcacacaagaaaaaaatcaagatggttgacacactgagtgaagagaacatcattggatacttcaagaacaagagcatcctcatcactggatcaacaggctttcttggaaagatactggtggagaagatactgagagttcaacctgatgtgaagaagatctacctcccggtgcgagcggtggatgccgcggcggcaaagcatcgggtggagactgaggtggtagggaaggagttgttcgggcttctgagggagaagcacgggggcaggtttcaatctttcatctgggaaaagatcgtcccattggccggagacgtgatgcgcgaggacttcggcgtcgacagcgagaccctgagggagctccgggtgacccaggagctcgatgtcatcgttaatggcgccgccaccaccaacttctacgaaaggtatgatgtggctctagacgtgaacgtgatgggagtgaagcatatgtgcaacttcgccaagaagtgccccaatctcaaggtgctcctccatgtctccacggcttacgtggcgggtgagaagcaagggctggtgcaagagagaccattcaagaatggcgagacgctgctcgaggggacccgcctcgacatcgacactgagctgaaactggccaaggacctgaaaaagcagcttgaggccgacgttgattcgtcgcccaaggccgaaaggaaggccatgaaggatcttggccttaccagggcccggcacttcaggtggccaaacacatacgtgttcaccaagtcgatgggggagatggtgctaagccagttgcagtgtgatgtccccgttgtcatcgtccgtcccagcatcatcacaagtgtccagaacgacccactgcccggatggatcgaaggcaccaggacgatcgacacgatcgtgatcggctatgcgaagcagaacctgacatacttcttggccgacctcaacctcaccatggatgtgatgccgggcgacatggtggtgaatgcgatgatggcggcaatagtggcacacagctcgtcctcattggagaagacaaagtcacatcccaagcaacatgcaccggcggtgtaccacgtgagctcgtcgctgcgtaatccggcaccatacaatgtgcttcatgaggctgggtttcggtacttcacggagcaccctcgcgtgggccctgacggtcgcaccgtgcgtacccataagatgacattcctcagcagcatggcttccttccacctatttatgatgctcaggtaccgcctcctcctcgagctcctccacctgctctccatcctctgctgcggcctcttcggcctcgacaccctctaccacgaccaagcacgcaagtacaggttcgtgatgcacctggtggatctgtacgggccctttgcgctgttcaaggggtgcttcgatgacgtcaacctaaacaagctcaggctcgccatgaccagcaaccatggtagcctcttcaatttcgacccgaagaccattgattgggacgagtacttctacagggtccacatccccggggtcataaagtacatgctcaagtgaaatatccgtgcaccgaaattcaggcgttgccttaataattaaataatcgtacgattgtaagaaatgtcctcccaaattgggttctaaccgttaaataatctatatgaaagaggaaaatgtgtctgtcgcttgatagagcaggcatcaacgttgcataagtttcttgaagacaaggaatgctacattatgtagtcccattctggttcatgtattgtattgctaattatgactagtattgattgttgttaccaagccgaattccagcacactggcggccgttactagtggatccgagctcggtaccaaaaaaaaaaaaaaaaaaaaaa

SEQ ID NO: 2 - protein, 507 aa, Triticum aestivum
MVDTLSEENIIGYFKNKSILITGSTGFLGKILVEKILRVQPDVKKIYLPVRAVDAAAAKHRVETEVVGKELFGLLREKHGGRFQSFIWEKIVPLAGDVMREDFGVDSETLRELRVTQELDVIVNGAATTNFYERYDVALDVNVMGVKHMCNFAKKCPNLKVLLHVSTAYVAGEKQGLVQERPFKNGETLLEGTRLDIDTELKLAKDLKKQLEADVDSSPKAERKAMKDLGLTRARHFRWPNTYVFTKSMGEMVLSQLQCDVPVVIVRPSIITSVQNDPLPGWIEGTRTIDTIVIGYAKQNLTYFLADLNLTMDVMPGDMVVNAMMAAIVAHSSSSLEKTKSHPKQHAPAVYHVSSSLRNPAPYNVLHEAGFRYFTEHPRVGPDGRTVRTHKMTFLSSMASFHLFMMLRYRLLLELLHLLSILCCGLFGLDTLYHDQARKYRFVMHLVDLYGPFALFKGCFDDVNLNKLRLAMTSNHGSLFNFDPKTIDWDEYFYRVHIPGVIKYMLK

SEQ ID NO: 3 - DNA, 1949 bp, Triticum aestivum
  CDS (74)..(1642)
ctctcttcttcctccgtctctcattctctcctcccgagcttcatagctcacacgcacacaaaaccaaggcaagatggtgggcacgctggatgaggggaagatcgtcgactacttcaggaacaagagcgtgctcatcaccggagccacgggattccttggcaagataatggtggagaagatcctgcgggtccagccggacgtgaagaggatctacctgccggtgcgggcggccgacgccgcggcggcgaggcgccgggtggagaccgaggtggtggggaaggagctgttctgcgtgctgcgggagcgccacggcgccgggttcgacgccttcgtcgccgacaaggtggtggggctggccggcgacgtcatgcgcgagggcttcggcgtcgaccccgccacgctgcgggacctccggctcgccgacgagctcaacgtcatcgtcaacggcgccgccaccaccaacttctacgaaaggtacgacgtggccctggacgtgaacgtggtgggggtgaagcacatgtgcgacttcgcccggaggtgccccaacctcgaggtgctcatgcacgtctccacggcctacgtcgccggcgagaagcaggggctggttccggagaggccgttcagggacggcgagacgctgcgcgacgacggcacccaactcgacatcgacgccgagatgaggctggccaaggacctcaggaagcagatggaggccgacgacgatgtggaccccaaggcccagaggaaggccatgaaggacctcggcctcaccagggccaggcactttgggtggcccaacacgtacgtgttcaccaaatccatgggggagatgatgctggcccagatgatgcgcgggggcgacgtgcccgtcgtcatcgtccggcccagcatcatcaccagcgtccagaacgacccactgccaggatggatcgaaggcaccaggacgatcgacgcaatcctgatcgggtacgcgaagcagagcctgtcgtgcttcctcgccgacctcgacctaaccatggacgtgatgcccggcgacatggtggtgaacgcgatgatggcggccacggtggcacatgcctcctccactcagacatcagagccagagaagaagccgcctccgcagcagcaacaccctcactcggtgccggcagcgccaacggtgtaccacgtgagctcgtcgctgcggcacccggctccgtacgcggtgttgtaccgaacggggatccggtacttcgaggagcacccacgggtggggcctgatggccgccccgtgcgcacccgtaaggtgcggttcctcggcagcatcgcggcgttccacctattcatggtgctcaagtaccgtgtcccccttgaactcctccgcctgctctccatcctctgttgcggcctctttggccttgccgccctctaccacgacctcgcccgcaagtacaggttcgtgatgcagctggtggacctgtacgggcccttctcgctcttcaagggttgcttcgacgatgtaaacctcaacaagctcaggctcgccatggccgacggtgaccatgccgattccgcattcaactttgaccccaagaccattgactgggacgactacttcttcaaggtccacatccctggtgtcatgaagtacgtccacaagtgatgttctgtgtgcgatctgcttctgcgtgctgagaaggaatggaggaaatcaaattaatggtagcgctagtgtgccttgcttgtgttgtgtaacctccttcttcgttcatcgagtattattggttgagtattgattgtattgtcattggaagttaaattaaccagtgactatgagtataactaagatgaaattacttgcatcatggcgggtctctaaaactaagatagtacaaggatcctatgaagtacattgaaattacttagtacttttcatggtactatcataatacaaaaaaaaaaaaaaaaaa

SEQ ID NO: 4 - protein, 522 aa, Triticum aestivum
MVGTLDEGKIVDYFRNKSVLITGATGFLGKIMVEKILRVQPDVKRIYLPVRAADAAAARRRVETEVVGKELFCVLRERHGAGFDAFVADKVVGLAGDVMREGFGVDPATLRDLRLADELNVIVNGAATTNFYERYDVALDVNVVGVKHMCDFARRCPNLEVLMHVSTAYVAGEKQGLVPERPFRDGETLRDDGTQLDIDAEMRLAKDLRKQMEADDDVDPKAQRKAMKDLGLTRARHFGWPNTYVFTKSMGEMMLAQMMRGGDVPVVIVRPSIITSVQNDPLPGWIEGTRTIDAILIGYAKQSLSCFLADLDLTMDVMPGDMVVNAMMAATVAHASSTQTSEPEKKPPPQQQHPHSVPAAPTVYHVSSSLRHPAPYAVLYRTGIRYFEEHPRVGPDGRPVRTRKVRFLGSIAAFHLFMVLKYRVPLELLRLLSILCCGLFGLAALYHDLARKYRFVMQLVDLYGPFSLFKGCFDDVNLNKLRLAMADGDHADSAFNFDPKTIDWDDYFFKVHIPGVMKYVHK

SEQ ID NO: 5 - DNA, 1949 bp, Triticum aestivum
  CDS (94)..(1617)
ctccttcttcctctctctctctctctctccctgctctccctgctctctctctctctctctctctctcttgcgcacacaagaaaaaaaatcaagatggttgacacactgagtgaagagaagatcattggatacttcaagaacaagagcatcctcatcactggatcaacaggctttcttggaaagatactagtggagaagatactgagagttcaacctgatgtaaagaagatctatctcccggtgcgagcggtggatgccgcggcggcgaaggatcgggtggagactgaggtggtagggaaggagttgttcgggcttctgagggagaagcacggggactggtttcaatctttcatctgtgaaaagatcgtcccattggccggagatgtgatgcgtgaggactttggcgtcgacagcgagaccctgagggagctccgggtgacccaggagctcgatgtcatcgttaatggcgccgccaccaccaacttctacgaaaggtatgatgtggccctggacgtgaacgtgatgggagtgaagcatatgtgcaacttcgccaagaagtgccccaatctcaaggtgctcctccatgtctccacggcttatgttgcgggtgagaagcaaggactcgtgcaagagagaccattcaagaatggcgagacgctgctcgaggggacccacctcgatatcgacaccgagctgaaactggccaaggacctgaaaaagcagcttgaggccgacgccgactcgtcgcccaagtcccaaaggaaggccatgaaggaccttggcatcaccagggcccggcacttcgggtggccgaacacatacgtgttcaccaagtcgatgggggagatggtgctgggccagttgaagtgtgatctccctgttgtcatcgtccgtcccagcatcatcaccagtgtccagaacgacccactgcccggatggatcgaaggcaccaggacgatcgacacgatcgtgatcggctatgcgaagcagaacctgacatacttcttggcggacctcaacctcaccatggatgtgatgccgggcgacatggtggtgaatgcgatgatggctgccatcgtggcgcacagctcgtccttattggagaagacacagtcacatcccgagccacacgcaccggcggtgtaccacgtgagctcgtcgcggcgtaacccggcgccgtacaatgtgctgcacgaggctgggtttcggtacttcacggagcaccctcgggtgggccctgacggccgcacggtgcgcacccataagatgacattcctcagcagcatggcttccttccacctctttatgatgctcaggtaccgcctcctccttgagctcctccacctgctctccgtcctctgttgtggcctcttcggcctcgacaccctctaccacgaccaagcacgcaagtacaggttcgtgatgcacctggtggatctgtatgggcccttcgcgctgttcaagggctgcttcgatgacgtcaacctaaacaagctcaggctcgccatgaccagcaaccatggaagcctctttaatttcgaccccaagaccattgactgggacgattacttctacagcgtccacatccccggggtcctaaagcacatgctcaactgaaatatccatgcaccgaaaatttaggcgttgccttaataattaaataatcgtacgttgtaagaaatatcctcccaaattgggttctaaccgttaaataatctgtgtgaaagagggaaatgtgtctgtcgcttgatagtgcaggcatcaacttgcatatgcttcttgaagaacaaggaatgctacatcatgtagtttcgttctggctcgtgctgtattgccaattatgactagtattggttgataacaatttcttgttcagtcacatattgtaccttgactagtattgattgttaattataatttcttttcaagagaaaaaaaaaaaaaaaaaa

SEQ ID NO: 6 - protein, 507 aa, Triticum aestivum
MVDTLSEEKIIGYFKNKSILITGSTGFLGKILVEKILRVQPDVKKIYLPVRAVDAAAAKDRVETEVVGKELFGLLREKHGDWFQSFICEKIVPLAGDVMREDFGVDSETLRELRVTQELDVIVNGAATTNFYERYDVALDVNVMGVKHMCNFAKKCPNLKVLLHVSTAYVAGEKQGLVQERPFKNGETLLEGTHLDIDTELKLAKDLKKQLEADADSSPKSQRKAMKDLGITRARHFGWPNTYVFTKSMGEMVLGQLKCDLPVVIVRPSIITSVQNDPLPGWIEGTRTIDTIVIGYAKQNLTYFLADLNLTMDVMPGDMVVNAMMAAIVAHSSSLLEKTQSHPEPHAPAVYHVSSSRRNPAPYNVLHEAGFRYFTEHPRVGPDGRTVRTHKMTFLSSMASFHLFMMLRYRLLLELLHLLSVLCCGLFGLDTLYHDQARKYRFVMHLVDLYGPFALFKGCFDDVNLNKLRLAMTSNHGSLFNFDPKTIDWDDYFYSVHIPGVLKHMLN

SEQ ID NO: 7 - DNA, 3392 bp, Triticum aestivum
cctctttaagtcggtcaggtacgtagcaaataaaccaaacaagcaggacatgtttgaaaatcactgttctaactttaataatgcacacaaggaattgaaggcttgataaatcctgtgctagtaccacccatccgactgtcggtttttaagacatggtagagtagagtactaacataacgatcttcaagatattgtgtccaagttctttgccacaggtttgtcgtattgagccaaaatataccactgaattatgtgctaatagtgtaattattggtcaattttttgttctaacgagagaaaaatacgctatgcgttttgaagggatggagtacgatgatactactctattgtactccacggatcaattgtaggagtaccctctaatttctaatggtataactttgatgacatacgatttgtattacgttggttgaattgtacatgggccttctataaagcgaaaaagaaaggaatactgagcttctctagttttctataamccagagttagacgtcctactggacaactcattcctaagtgtgtagcaataatactgagtactaccggttgtccacttgagctattaatttgatggctagcaacgtactgcaaccccaataataaccctgtttgaaaatctagagtacctctacgcgttattttgactgcatgtgattcctctcatgatcaccaggtcccttgktttgggttggacagcgcataaatgttgcgctcaagtgcctcaaattaagactcccctctcctacctacaacacaaaagacctttttttcagcaaaagagggcgccaccccctccgatttcatataatgaaaccaactggttcaactgccaaacaaacaacaaaaacaaaacgaaacaccttcatggcccgtgtataaatattgtgtacatgaccctctttccactcacccacacaaatcatctcctctctctctctctctctctctctctctcgcacgcacgcacgcacgcacacaagaaaaaaatcaagatggttgacacactgagtgaagagaacatcattggatacttcaagaacaagagcatcctcatcactggatcaacaggctttcttggaaagagtatgcatgcatgcataatgtttatgcacacatattacgcatgcatatacttttttttggcaaattctagaagttttttgcgtcaagttcatatgtgctgtcctcatctgttaattagttcactgttggtgtgcgtatatgcagtactggtggagaagatactgagagttcaacctgatgtgaagaagatctacctcccggtgcgagcggtggatgccgcggcggcaaagcatcgggtggagactgaggtagtgttgtccatcatggttcttcatgatctataccctttacccctctcatgattcttttgatcccttttcttcagagttagcgctgactaatttcttttctatatgcgcaggtggtagggaaggagttgttcgggcttctgagggagaagcacgggggcaggtttcaatctttcatctgggaaaagatcgtcccattggccggagacgtgatgcgcgaggacttcggcgtcgacagcgagaccctgagggagctccgggtgacccaggagctcgatgtcatcgttaatggcgccgccaccaccaacttctacgaaaggtgcgtcattgtcaactgattgttttgtccaagaaaggaaaaatcagcagagaacttgagtaggtgcagcaagagtacgtacgcagtgaatggtctcagaagaacacttgtgtgcgtgcaggtatgatgtggctctagacgtgaacgtgatgggagtgaagcatatgtgcaacttcgccaagaagtgccccaatctcaaggtgctcctccatgtctccacgggtacgtacaccaactctacaggtaaaaacaaaaataaagttcttggatgttaattataatacacctagatttgatttacaaatgaagttaataaattcatatatgagttggtgcagcttacgtggcgggtgagaagcaagggctggtgcaagagagaccattcaagaatggcgagacgctgctcgaggggacccgcctcgacatcgacactgagctgaaactggccaaggacctgaaaaagcagcttgaggccgacgttgattcgtcgcccaaggccgaaaggaaggccatgaaggatcttggccttaccagggcccggcacttcaggtggccaaacacatacgtgttcaccaagtcgatgggggagatggtgctaagccagttgcagtgtgatgtccccgttgtcatcgtccgtcccagcatcatcacaagtgtccagaacgacccactgcccggatggatcgaaggcaccaggttcattatatgtttcttgtcctttctctgcctccaaatttagagtgcaatcgtcttactctgttgcaaatgccaaaagaagtaaaatatgatatttgttcaatgtaaaaatgtaaattgcaggacgatcgacacgatcgtgatcggctatgcgaagcagaacctgacatacttcttggccgacctcaacctcaccatggatgtggtaagcaacgttgtactatgcatgcagttaagatatattccaggcaatggttggttgtcagtccagtccaggaatccgtacagtaagatgaatttcgacgacgatggtgaaagcaatcgtttggttgggtatatgttgctgtagatgccgggcgacatggtggtgaatgcgatgatggcggcaatagtggcacacagctcgtcctcattggagaagacaaagtcacatcccaagcaacatgcaccggcggtgtaccacgtgagctcgtcgctgcgtaatccggcaccatacaatgtgcttcatgaggctgggtttcggtacttcacggagcaccctcgcgtgggccctgacggtcgcaccgtgcgtacccataagatgacattcctcagcagcatggcttccttccacctatttatgatgctcaggtaccgcctcctcctcgagctcctccacctgctctccatcctctgctgcggcctcttcggcctcgacaccctctaccacgaccaagcacgcaagtacaggttagttagttggttgaaatcttgtgcggttgtatcttcttgatggctcccacataattaagatgacacgacttttattgttgtattgttataggttcgtgatgcacctggtggatctgtacgggccctttgcgctgttcaaggggtgcttcgatgacgtcaacctaaacaagctcaggctcgccatgaccagcaaccatggtagcctcttcaatttcgacccgaagaccattgattgggacgagtacttctacagggtccacatccccggggtcataaagtacatgctcaagtga

SEQ ID NO: 8 - DNA, 5848 bp, Triticum aestivum
ctgaccaaacatctcccatcaggaaccatgttactatttaattttgcatgttctttgcttcgtgctgtttgtcaaggacacgctagcttctgcattatacagacaggctggaaatctgtaaataatgtgttggtgaatctttggagtatttttctaatgttgatcagtacttgttgacctaggtggcgactaactttggacaggggcaagcaagcaaaagcagagacagctagcttctgcattatcagaaaatagcctgtgttggtgaatcttcagagtgctcctactcccccgtccccccaaatgttcagtacttgttgacctactggagctggacgacccgctctgaaagttttacttgtccacacttgtctggccaaaatccccaatgaatcagaatataggctgaaaaatgagtgggtcgtttttggagctcggcctccacggaggccgaaaaaaaactcaaaattctaatttctcagtttaaaaaaaaatgaaaaaaattgtacaagtaaacaaggttgtgatgtgtatgtgtgtaaaatttcagaacaaaatactttgaaatgcgatctgagcaaaaaaaaacaaattcatagacttttgagaatgaatagtgcaagctactaaaacgcatgagattctttttgcgtgtagcactcattttaatgtatttcgtcctgaaaattacaaacttgtgcattatgtctataagtatatggtggatttaaaaaaaaactgaaacatgcaaatttgaattttgatcatttcaaaatcggcctccatggagcatttttgctgaaaaatctaaaagaaaacgcagtccaaccttagtaattacccatgttggtgaatccagagtgtttacgataaatataggacctctttagctagcgaacgatggcatttgcgaatgttatttatggcaaatccttcagaacacacatgccaatttcttcataaacacatggcaattctggcaaatattttgctgtgaaaattgttgtgcttgcttcaaggaattgccatgttcccgcaatataaaatgccatgaaaaacgttcattttgtcatatccccagcgaacgtccgccgagtagcattaccgtaaatatagggggtgtttggttcagaagttctaggactttttctagtcccagggactaatcaaaaaagactctctaatagagtctttttctagtccctatagaaaaagtccctcccgtttggttcctagggactttttttagtctctaggactaaaaagtccctgaaccaaacaccccatattagtagtaccgcaaaaagtagtaagtactacagtattggtggatctttaatgagttaattaacctagaatggctcctgctcctccggtttcactggaaaagcaggttgtggttgggaagcacaagcgtgttgccgttgctaattacgtgcactcaatagccagccagccaccctcaacacctcgctggccggccctgcgcgtgtataaatactgggtgccggagcacacaagcctcacacaccaagcacacctctcttcttcctccgtctctcattctctcctcccgagcttcatagctcacacgcacacaaaaccaaggcaagatggtgggcacgctggatgaggggaagatcgtcgactacttcaggaacaagagcgtgctcatcaccggagccacgggattccttggcaagagtaagtgagcatgatgaggatatatcatatatgcatgccttgctattttctgaagaacagttcatgcatccatgctgagagtccttggtgttgcattgtgtgctcactcttcgacctgtgcagtaatggtggagaagatcctgcgggtccagccggacgtgaagaggatctacctgccggtgcgggcggccgacgccgcggcggcgaggcgccgggtggagaccgaggtgcgtgcctctctgcccgtccatcatgctccagcatgatggtgatccttgcaccccctccatcaatctcgctctgaacactcctctgttctagagtaatactagtagtagacaaaggatgcatgccggcagcatggttctcatgctgcaaggaacacatgcgagctttctctcagacagagaagcccccaagctgctcccactgtgccccttgtgtctcttcattccgtttcatattcactactctttttttcattgataatcaatactgtaatactagtaatattatgtggcgcatcggaacaaaacatgcatagatatcacagaaccataaatagtccagctgcccgctgctatatcagtgatgcaaacatgaacatgggtagaatagttatcttataggcataaaccggcattagtcaggactaggtgcacactgttgagtctcatccctgccgaacattcaggcaggtcacatgccattttttcttctctttctgaggttgagagtcacatgccatactctgcatgagaggtggccatgaataatctcagctgtacaccgctcattcttctctactactccctccgttcctaaatatgtctttttaaagattccaacaagtgactacatacagaataaaatgaatgaatctatattctaaaatatgtctacatacatccatatgttatagtccatttaaaatgtctaaaaaaacttatatttaggaacggagggagtagatactagttaaatgcacaaatattctgccagcttttggtatgccaggattaatttgaacgagaaaccagatactagtaggcgtatggttgacgaaattttcaacattttcatttacgcccgatcctggaaaccgggaaccttgggaaaaccggccgcaggatgacaattaaaccggcggcgaagtagcgcagctgtgtgtgtatccctaggagtcgtataggagttatggtgtgggtaggccggtaggtttggtcaggtcgactcgattcacaagttttcaatggcctctgccgcatgcagtgcacaactggtactgcaccaccacctagctagctagccggccagtgtgcagcctgcatgcatgcatgcatgcatggctggctggctgctgctgggaaatgacgagcgcagggcaccatctggcactggcaccggcaccactaactacagcttcatcaacttgagaattcagaacccagagtttcttgtcatatgtgtatagagtggaccgcaagtctgcaatgatgtgttggtacctgattgcatcaacaatgaacacatataactgcaattatgttttggttagtagattctagtatattgcaaaagaaaaaaagtgtagttattgagtggtgccaaggagatccagagcagagacaatgggcagtatgcttggtcctccaccctactcctactccatctagtaaaagccgggaaagaaacccttccagcacgcctggcccctcttcatttcttcatatgcattcactgatccttcattgttcggcatgggcaatttacagctgcagacagttgcacgggtcggttgtcgtgcgctgcagaagcaatttatagtttttaccaaaagttgcctgagacactcaccatgttgggtggtggatccacagtgcgtgcatgcatgtatcaaacgcacattgcaggttaagaattggtgtaaatcaagcacgtctattgatcaaactaattttatcgagaccggcgtggttcgcaggtggtggggaaggagctgttctgcgtgctgcgggagcgccacggcgccgggttcgacgccttcgtcgccgacaaggtggtggggctggccggcgacgtcatgcgcgagggcttcggcgtcgaccccgccacgctgcgggacctccggctcgccgacgagctcaacgtcatcgtcaacggcgccgccaccaccaacttctacgaaaggtgctgtgtactattgactgttttaattaacttcgtcagagagaaaattcagcgggagagaacgtgcatggctgcaccttcttctccagagtagtgagctcatcaatggcctctgcaactaactttggaatggacgtgtgcgtgctcaggtacgacgtggccctggacgtgaacgtggtgggggtgaagcacatgtgcgacttcgcccggaggtgccccaacctcgaggtgctcatgcacgtctccacggcctacgtcgccggcgagaagcaggggctggttccggagaggccgttcagggacggcgagacgctgcgcgacgacggcacccaactcgacatcgacgccgagatgaggctggccaaggacctcaggaagcagatggaggccgacgacgatgtggaccccaaggcccagaggaaggccatgaaggacctcggcctcaccaggttagcaatcctatatttttccaaagtttctcctctttcctgtgaaattcaagttcaaaaaacagagggttttattttgtgaaacttggaactgaaattcaatatatttttttgtaattgtgtgtgcagggccaggcactttgggtggcccaacacgtacgtgttcaccaaatccatgggggagatgatgctggcccagatgatgcgcgggggcgacgtgcccgtcgtcatcgtccggcccagcatcatcaccagcgtccagaacgacccactgccaggatggatcgaaggcaccaggtcggttcaaactctttgggtctaaatatgaatgccaagtttcaaattcaaattctaacaccgaaatgaaaaatgcaggacgatcgacgcaatcctgatcgggtacgcgaagcagagcctgtcgtgcttcctcgccgacctcgacctaaccatggacgtggtaattgcttcactcgttattagttcagcaaatgcagagctgctagtgctactgtcttcttgcgaacgtgctgcagtaatgagtaataagtatactagtacagtgtgtgaccatgctgcagtaagttgcatctgaagcgtctgtgccaaccaggccaagtggctgctgcagtgcatcagccaaccttagatgaacttggctgcgtgcggttactatctgaatcttgcctgttacagcaaggttacatatgctgctactggattatcaagttctaacccaaatgcgctaccatggtgtctgcagatgcccggcgacatggtggtgaacgcgatgatggcggccacggtggcacatgcctcctccactcagacatcagagccagagaagaagccgcctccgcagcagcaacaccctcactcggtgccggcagcgccaacggtgtaccacgtgagctcgtcgctgcggcacccggctccgtacgcggtgttgtaccgaacggggatccggtacttcgaggagcacccacgggtggggcctgatggccgccccgtgcgcacccgtaaggtgcggttcctcggcagcatcgcggcgttccacctattcatggtgctcaagtaccgtgtcccccttgaactcctccgcctgctctccatcctctgttgcggcctctttggccttgccgccctctaccacgacctcgcccgcaagtacaggtacattcttcttccggaattagttgacgctcaaacgaattatctagagggagcacagtttaaacacttttgcggttgtaccgtgttgatgttataccattggcttaagaacatgcattttgtgtaggttcgtgatgcagctggtggacctgtacgggcccttctcgctcttcaagggttgcttcgacgatgtaaacctcaacaagctcaggctcgccatggccgacggtgaccatgccgattccgcattcaactttgaccccaagaccattgactgggacgactacttcttcaaggtccacatccctggtgtcatgaagtacgtccacaagtga

SEQ ID NO: 9 - DNA, 2800 bp, Triticum aestivum
actccgacacaatattttgactgcatgtgattcctcttatgatcactgagtctttgtttcggttggacagcgcataaatgttgcgctcaagtgcctcaaattaagacacccctcttctacctacaacacgaaagacccttttttcttagcaaaaatttcatacaatgaaaccaactggttcaactgccaaaacaacagactccaagacaaaacaccttgatggcccgtgtataaatattgtgtacaggagcctctttccactcacctacacaaatcatctccttcttcctctctctctctctctccctgctctccctgctctctctctctctctctctctctcttgcgcacacaagaaaaaaaatcaagatggttgacacactgagtgaagagaagatcattggatacttcaagaacaagagcatcctcatcactggatcaacaggctttcttggaaagagtatgcatgcatgcacgcataatgtttatacaaacatattacgcatgcatatactatttgttgcaaattctagaagttttttttttgcgtcaagttgatatgtgttgtgctcatctgttaattagttctactgttggtgcatatgtgcagtactagtggagaagatactgagagttcaacctgatgtaaagaagatctatctcccggtgcgagcggtggatgccgcggcggcgaaggatcgggtggagactgaggtagtgttgtccatcatggttcttcatgatctagacctctactgctctcatgattcttttgatccctttgcttgagtgttggtactgaataatttattttctatgtgcccaggtggtagggaaggagttgttcgggcttctgagggagaagcacggggactggtttcaatctttcatctgtgaaaagatcgtcccattggccggagatgtgatgcgtgaggactttggcgtcgacagcgagaccctgagggagctccgggtgacccaggagctcgatgtcatcgttaatggcgccgccaccaccaacttctacgaaaggtgcgtcgtcgtcaactgattgttttgtccaagaaaggaaaaaaaatcagcagagaacttgagtaggtgcagcaagagtacggagtggaattgtgtgcgtgcaggtatgatgtggccctggacgtgaacgtgatgggagtgaagcatatgtgcaacttcgccaagaagtgccccaatctcaaggtgctcctccatgtctccacgggtacgtacaccaactctacaaattaccatcatggactatgaacttggatgcttctggggaaaacaaaaatgaagttcttggatgtaattaaagtacacctagatttgatttacaaatcaagttaatgaattcatacatgagttggtgcagcttatgttgcgggtgagaagcaaggactcgtgcaagagagaccattcaagaatggcgagacgctgctcgaggggacccacctcgatatcgacaccgagctgaaactggccaaggacctgaaaaagcagcttgaggccgacgccgactcgtcgcccaagtcccaaaggaaggccatgaaggaccttggcatcaccagggcccggcacttcgggtggccgaacacatacgtgttcaccaagtcgatgggggagatggtgctgggccagttgaagtgtgatctccctgttgtcatcgtccgtcccagcatcatcaccagtgtccagaacgacccactgcccggatggatcgaaggcaccaggttcgttatatgcttctctttcctttccctgcctctaaatttaaagtgcaatcgttttaatctgttgcaaatgcaagtaaataaaggatgtttgttcaatgtaaaaatgtaaattgcaggacgatcgacacgatcgtgatcggctatgcgaagcagaacctgacatacttcttggcggacctcaacctcaccatggatgtggtaagcaacgttgcactatgcatgcagttaattaacatatattccaggcatgcaatggttggttgtcagtccaggaatccatacagtaagatatggatttcaacgatggcggtgaatgcaatcgtgtggttgggtatatgttggtgcagatgccgggcgacatggtggtgaatgcgatgatggctgccatcgtggcgcacagctcgtccttattggagaagacacagtcacatcccgagccacacgcaccggcggtgtaccacgtgagctcgtcgcggcgtaacccggcgccgtacaatgtgctgcacgaggctgggtttcggtacttcacggagcaccctcgggtgggccctgacggccgcacggtgcgcacccataagatgacattcctcagcagcatggcttccttccacctctttatgatgctcaggtaccgcctcctccttgagctcctccacctgctctccgtcctctgttgtggcctcttcggcctcgacaccctctaccacgaccaagcacgcaagtacaggttagtcggtttaaatcttttgcggatggcatttttgataacaagtatttccggacggagggagtatcttcttgatggcgcggcatatatgatgacacggcctttattgttatattgttgtaggttcgtgatgcacctggtggatctgtatgggcccttcgcgctgttcaagggctgcttcgatgacgtcaacctaaacaagctcaggctcgccatgaccagcaaccatggaagcctctttaatttcgaccccaagaccattgactgggacgattacttctacagcgtccacatccccggggtcctaaagcacatgctcaactga

SEQ ID NO: 10 - DNA, 20 bp, Artificial sequence
  OL2707 primer
gactacgtcgtccaaggccg

SEQ ID NO: 11 - DNA, 21 bp, Artificial sequence
  OL2708 primer
gtcgaactgcttgagcagcgc

SEQ ID NO: 12 - DNA, 19 bp, OL2881 primer
gcagaacctgacatacttc

SEQ ID NO: 13 - DNA, 18 bp, Artificial sequence
  OL2885 primer
gaggcggtacctgagcat

SEQ ID NO: 14 - DNA, 18 bp, OL2884 primer
ttcgcatagccgatcacg

SEQ ID NO: 15 - DNA, 18 bp, Artificial sequence
  OL2883 primer
aatgccggccctggtaag

SEQ ID NO: 16 - DNA, 18 bp, Artificial sequence
  OL2880 primer
caggtggccaaacacata